(19) United States
(12) Patent Application Publication
(10) Pub. No.: US 2022/0146536 A1
(43) Pub. Date: May 12, 2022

(54) METHODS, COMPOSITIONS AND KITS FOR THE ASSESSMENT OF MILD COGNITIVE IMPAIRMENT

(71) Applicant: INSTITUT NATIONAL DE LA RECHERCHE SCIENTIFIQUE, Quebec (CA)

(72) Inventors: Charles RAMASSAMY, Laval (CA); Morgane PERROTTE, Caen (FR); Mohamed HADDAD, Laval (CA)

(21) Appl. No.: 17/594,238

(22) PCT Filed: Apr. 9, 2020
(86) PCT No.: PCT/CA2020/050475

§ 371 Date: Oct. 7, 2021

Related U.S. Application Data
(60) Provisional application No. 62/831,772, filed on Apr. 10, 2019.

(51) Int. Cl. G01N 33/68

(52) U.S. Cl. CPC G01N 33/6896

(57) ABSTRACT

Methods, compositions and kits for the assessment and management of mild cognitive impairment (MCI) and early stage Alzheimer's disease (AD), and to monitor changes in cognitive functions in subjects over time, are described. The assessment for MCI and early stage AD is based on the level of BDNF, NSE, S100B, PGRN and/or the PGRN/BDNF ratio, in plasma-derived extracellular vesicles (EVs) from a subject.

### CROSS REFERENCE TO RELATED APPLICATIONS

The present application claims the benefit of U.S. provisional application Ser. No. 62/831,772 filed Apr. 10, 2019, which is incorporated herein by reference.

### TECHNICAL FIELD

The present disclosure generally relates to the field of neurocognitive disorders, and more particularly to the assessment and management of mild cognitive impairment (MCI) and Alzheimer's disease (AD).

### BACKGROUND ART

Alzheimer's disease (AD), a multifactorial disorder, is the most common type of dementia and is characterized clinically by progressive cognitive decline and neuropathologically by synaptic and neuronal loss and the presence of amyloid plaques. During the progression of the disease, some alterations occur in the brain like a deficiency of cellular survival factors, an inflammation and a metabolic disorder (1-4).

The development of AD pathogenesis is insidious, and no clear event defines the onset of the disease (5). Hence, the detection of the disease at the early stages is a considerable challenge. The prodromal stage of dementia, mild cognitive impairment (MCI), provides an important opportunity for potential intervention to prevent the onset of dementia. However, the current standardized criteria for the assessment of MCI and AD including cognitive changes, abnormal cerebrospinal fluid (CSF) levels of pathogenic proteins, and MRI and PET bioimaging data, have some limits (6). The clinicopathologic heterogeneity, the high costs of imaging and the invasive nature of CSF collection limit their usefulness for routine clinical testing. Thus, there is a strong necessity to identify non-invasive blood biomarkers easily measurable that could facilitate early and accurate assessment, as well as to evaluate the therapeutic efficacy of new treatment. Despite intense research in the field, there is no peripheral biomarker that has got beyond the discovery stage. Early identification of subjects suffering from MCI, prior to overt symptoms of AD, would allow for earlier onset of treatment to prevent or delay AD.

There is thus a need for novel tools for the assessment and management of MCI and AD.

The present description refers to a number of documents, the content of which is herein incorporated by reference in their entirety.

### SUMMARY OF THE DISCLOSURE

The present disclosure provides the following items 1 to 47:

1. A method for identifying a subject suffering from mild cognitive impairment (MCI) or early stage Alzheimer's disease (AD) comprising determining, in a sample comprising extracellular vesicles (EVs), preferably plasma-derived extracellular vesicles (pEVs) from the subject, at least one of:
(i) levels of Brain-Derived Neurotrophic Factor (BDNF);
(ii) levels of Neuron Specific Enolase (NSE);
(iii) levels of S100 calcium-binding protein B (S100B);
(iv) levels of Progranulin (PGRN);
(v) a ratio of the levels of PGRN to BDNF (PGRN/BDNF ratio); and
(vi) levels of glyoxalase 1 (GLO-1)
wherein lower levels of BDNF in the sample relative to a reference BDNF level, lower levels of NSE in the sample relative to a reference NSE level, lower levels of S100B in the sample relative to a reference S100B level, lower levels of PGRN in the sample relative to a reference PGRN level, a lower PGRN/BDNF ratio in the sample relative to a reference PGRN/BDNF ratio, and/or lower levels of GLO-1 in the sample relative to a reference GLO-1 level, is indicative that the subject suffers from MCI or early stage AD.
2. The method of item 1, comprising determining the levels of BDNF.
3. The method of item 1 or 2, comprising determining the levels of NSE.
4. The method of any one of items 1 to 3, comprising determining the levels of S100B.
5. The method of any one of items 1 to 4, comprising determining the levels of PGRN.
6. The method of any one of items 1 to 5, comprising determining the PGRN/BDNF ratio.
7. The method of any one of items 1 to 6, comprising determining the levels of GLO-1.
8. The method of any one of items 1 to 7, wherein the reference BDNF level is about 72 pg/mL.
9. The method of item 8, wherein the reference BDNF level is about 58.1 pg/mL.
10. The method of any one of items 1 to 9, wherein the reference NSE level is about 403 pg/mL.
11. The method of item 10, wherein the reference NSE level is about 394 pg/mL.
12. The method of any one of items 1 to 11, wherein the reference S100B level is about 554 pg/mL.
13. The method of item 12, wherein the reference S100B level is about 549 pg/mL.
14. The method of any one of items 1 to 13, wherein the reference PGRN level is about 475 pg/mL.
15. The method of any one of items 1 to 14, wherein the reference PGRN/BDNF ratio is about 14,2.
16. The method of item 15, wherein the reference PGRN/BDNF ratio is about 4,1.
17. The method of any one of items 1 to 16, further comprising isolating the EVs from a biological sample prior to said determining.
18. The method of any one of items 1 to 17, wherein the method is for identifying a subject suffering from MCI.
19. The method of any one of items 1 to 17, wherein the method is for identifying a subject suffering from early stage AD.
20. A method for (a) preventing or delaying the onset of AD in a subject suffering from MCI, or (b) preventing or delaying the progression of AD in a subject suffering from early stage AD, the method comprising administering a therapy for AD to a subject suffering from MCI or early stage AD identified using the method of any one of items 1 to 19.
21. The method of item 20, further comprising performing the method of any one of items 1 to 19 to identify subject suffering from MCI or early stage AD.
22. Use of a therapy for AD for (a) preventing or delaying the onset of AD in a subject suffering from MCI, or (b) preventing or delaying the progression of AD in a subject suffering from early stage AD in a subject suffering from MCI or early stage AD identified using the method of any one of items 1 to 19.
23. The use of item 22, further comprising performing the method of any one of items 1 to 19 to identify said subject suffering from MCI or early stage AD prior to said use.
24. A kit for identifying a subject suffering from mild cognitive impairment (MCI) or early stage Alzheimer's disease (AD) comprising:
(a) reagents for determining the levels of BDNF, NSE, S100B, GLO-1 and/or PGRN; and
(b) instructions setting forth the method of any one of items 1 to 19.
25. The kit of item 24, wherein the reagents for determining the levels of BDNF, NSE, S100B and/or PGRN comprise an anti-BDNF antibody, an anti-NSE antibody, an anti-S100B antibody, an anti-GLO-1 antibody and/or an anti-PGRN antibody.
26. The kit of item 24 or 25, further comprising reagents for isolating extracellular vesicles from a plasma sample.
27. A method for identifying a subject suffering from early stage Alzheimer's disease (AD) comprising determining, in a sample comprising plasma-derived extracellular vesicles (EVs) from the subject, levels of glyoxalase 1 (GLO-1), wherein lower levels of GLO-1 in the sample relative to a reference GLO-1 level, is indicative that the subject suffers from early stage AD.
28. The method of item 27, further comprising isolating the EVs from a biological sample prior to said determining.
29. A kit for identifying a subject suffering from early stage Alzheimer's disease (AD) comprising: (a) reagents for determining the levels of GLO-1; and (b) instructions setting forth the method of item 27 or 28.
30. The kit of item 29, wherein the reagents for determining the levels of GLO-1 comprise an anti-GLO-1 antibody.
31. The kit of item 29 or 30, further comprising reagents for isolating extracellular vesicles from a plasma sample.
32. A method for detecting a change in cognitive function over time in a subject, comprising determining in a sample comprising plasma-derived extracellular vesicles (EVs) from the subject, at least one of: (i) levels of Brain-Derived Neurotrophic Factor (BDNF); (ii) levels of S100 calcium-binding protein B (S100B); (iii) levels of Progranulin (PGRN); (iv) levels of receptor for advanced glycation end products (RAGE); (v) levels of glial fibrillary acidic protein (GFAP); (vi) glyoxalase 1 (GLO-1): and (vii) a ratio of the levels of PGRN to BDNF (PGRN/BDNF ratio).
33. The method of item 32, wherein the method comprises measuring the levels of RAGE.
34. The method of item 32 or 33, wherein the method comprises measuring the levels of GFAP.
35. The method of any one of items 32 to 34, wherein the method comprises measuring the levels of GLO-1.
36. The method of any one of items 32 to 35, wherein the method comprises measuring the levels of BDNF.
37. The method of any one of items 32 to 36, wherein the method comprises measuring the levels of S100B.
38. The method of any one of items 32 to 37, wherein the method comprises measuring the levels of PGRN.
39. The method of any one of items 32 to 38, wherein the method comprises measuring the PGRN/BDNF ratio.
40. The method of any one of items 32 to 39, further comprising isolating the EVs from a biological sample prior to said determining.
41. A method for preventing or delaying a decline of cognitive function in a subject, the method comprising administering a therapy for improving cognitive function in a subject having a decline in cognitive function identified using the method of any one of items 32 to 42. The method of item 41, further comprising performing the method of any one of items 32 to 40 to identify said subject having a decline of cognitive function.
43. Use of a therapy for improving cognitive function for preventing or delaying a decline of cognitive function in a subject having a decline in cognitive function identified using the method of any one of items 32 to 40.
44. The use of item 43, further comprising performing the method of any one of items 32 to 40 to identify said subject having a decline of cognitive function prior to said use.
45. A kit for detecting a change in cognitive function over time in a subject comprising: (a) reagents for determining the levels of BDNF, S100B, PGRN, RAGE, GFAP and/or GLO-1; and (b) instructions setting forth the method of any one of items 32 to 40.
46. The kit of item 45, wherein the reagents for determining the levels of BDNF, S100B, PGRN, RAGE, GFAP and/or GLO-1 comprise an anti-BDNF antibody, an anti-RAGE antibody, an anti-S100B antibody, an anti-PGRN antibody, an anti-GFAP antibody and/or an anti-GLO-1 antibody.
47. The kit of item 45 or 46, further comprising reagents for isolating extracellular vesicles from a plasma sample.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 is a scheme showing the inclusion and exclusion criteria of the study described herein.

FIGS. 2A-D show the characterization of total extracellular vesicles isolated from plasma (pEVs). FIG. 2A: Transmission electron microscopy images revealed the characteristic shape and size of pEVs from a control participant. FIG. 2B: Particles concentrations were analyzed by Nanoparticle Tracking Analysis (NTA) using Nanosight NS300 system and were expressed with means (particle number/ml, black line)±standard deviation (dotted line), n=3. FIG. 2C: Western blot analysis of protein lysates from pEVs (15 pg respectively) probed for exosomal markers (TSG101 and CD63), cerebral markers (GFAP and L1CAM) and negative control (Calnexin). FIG. 2D: Total protein concentration of pEVs was measured with the bichinchoninic acid (BCA) assay in each group. Total proteins were also stained with Coomassie blue to compare the pattern of total protein content in plasma and pEVs.

FIGS. 3A-F show the levels of some brain derived proteins including BDNF, NSE, Progranulin and S100B (FIGS. 3A-D) and the ratio of Progranulin/BDNF (FIG. 3E) in pEVs. Each point represents the value for one patient or control subject. The mean±SEM for each group is shown by a horizontal line and is expressed in pg/ml (normalized using total protein concentration). For concentrations of BDNF, NSE and S100B and ratio of Progranulin/BDNF, statistical analysis was performed using the one way ANOVA followed by Tukey post hoc test (alpha=0.05) with ^{a}p<0.05, ^{aa}p<0.01 compared to healthy controls; ^{b}p<0.05 compared to MCI subjects; ^{c}p<0.05 compared to mild AD patients and ^{e}p<0.05, ^{ee}p<0.01, ^{eee}p<0.001 compared to severe AD patients. For Progranulin concentrations, statistical analysis was performed by the nonparametric Kruskal-Wallis test followed by Dunn's test (alpha=0.05) with ^{a}p<0.05 compared to healthy controls and ^{b}p<0.05, compared to MCI subjects. FIG. 3F: Scatter plots of pEVs Progranulin concentrations in relation to pEVs BDNF concentrations. Correlation coefficients (Pearson R and R²) and p values were determined using Pearson correlation. The confidence interval (CI) range was plotted in correlation plots (grey area).

FIGS. 4A-E show the results of receiver operating characteristic (ROC) curve analyses. The plot represents the performance of the Progranulin/BDNF ratio (FIG. 4A) and the levels of NSE (FIG. 4B), Progranulin (FIG. 4C), S100B (FIG. 4D) and BDNF (FIG. 4E) in pEVs to differentiate control subjects to MCI subjects. Area under the curve (AUC) values, standard errors (Sdt. Error), p values and 95% confidence intervals (CI) are indicated on the curve. ns, not significant.

FIGS. 5A-D show the results of ROC curve analyses. The plot represents the performance of levels of BDNF (FIG. 5A), NSE (FIG. 5B), the ratio of Progranulin/BDNF (FIG. 5C) and levels of S100B (FIG. 5D) in pEVs to differentiate control subjects to mild AD patients. Area under the curve (AUC) values, standard errors (Sdt. Error), p values and 95% confidence intervals (CI) are indicated on the curve. ns, not significant.

FIGS. 6A-H show the results of correlation analysis with cognitive performances and age. Scatter plots of pEVs Progranulin/BDNF ratio in relation to MoCA scores (FIG. 6A) or age (FIG. 6E), pEVs BDNF levels in relation to MMSE scores (FIG. 6B) or age (FIG. 6F), pEVs Progranulin levels in relation to MMSE scores (FIG. 6C) or age (FIG. 6G) and pEVs S100B levels in relation to MoCA scores (FIG. 6D) or age (FIG. 6H). Correlation coefficients (Pearson R and R2) and p values were determined using Pearson correlation. The confidence interval (CI) range was plotted in correlation plots (grey area).

FIGS. 7A-E show the results of ROC curve analysis. The plot represents the performance of the levels of BDNF (FIG. 7A), NSE (FIG. 7B), Progranulin (FIG. 7C) and S100B (FIG. 7D) and the ratio of Progranulin/BDNF (FIG. 7E) in pEVs to differentiate control participants to AD patients (all stages combined). Area under the curve (AUC) values, standard errors (Sdt. Error), p values and 95% confidence intervals (CI) are indicated on the curve.

FIGS. 8A-E show the results of ROC curve analysis. The plot represents the performance of the levels of BDNF (FIG. 8A), NSE (FIG. 8B), Progranulin (FIG. 8C) and S100B (FIG. 8D) and the ratio of Progranulin/BDNF (FIG. 8E) in pEVs to differentiate MCI subjects to AD patients (all stages combined). Area under the curve (AUC) values, standard errors (Sdt. Error), p values and 95% confidence intervals (CI) are indicated on the curve.

FIG. 9 is a graph depicting EVs RAGE levels in control subjects, MCI and different groups of AD patients. Each point represents the value for one patient or control subject. Difference between groups were analyzed with the one-way ANOVA followed by LSD test Values are mean±SEM with * P<0.05, ** P<0.01, *** P<0.001 versus LS AD patients. Abbreviations: AD: Alzheimer disease; ES: early stage of Alzheimer disease; MS: Moderate-stage of Alzheimer disease; LS: late-stage of Alzheimer disease; EVs: Extracellular vesicles.

FIGS. 10A-E show the assessment of serum and EVs GFAP levels from control, MCI and different AD groups by Western blot. FIGS. 10A and C: Representative Western blot of GFAP detection in serum (FIG. 10A) and in EVs (FIG. 10A). The Coomassie blue stained total proteins was used as the loading control for serum and EVs samples. FIGS. 10B and D: Quantitative results of the normalized of GFAP in serum (FIG. 10B) and in EVs (FIG. 10D) to their respective loading total proteins. Each point represents the value obtained from one patient or control subject. The difference between groups was analyzed with one-way ANOVA followed by the LSD post hoc test. FIG. 10E: Comparison between serum and EVs GFAP levels in different study groups. The difference in each group was analyzed with Student-t test. Values are mean±S.E.M with * p<0.05, ** p<0.01, *** p<0.001.

FIGS. 11A-E show the assessment of serum and EVs GLO-1 levels from control, MCI and different AD groups by Western blot. FIGS. 11A and C: Representative Western blot of GLO-1 detection in serum (FIG. 11A) and in EVs (FIG. 11A). The Coomassie blue stained total proteins was used as the loading control for serum and EVs samples. FIGS. 11B and D: Quantitative results of the normalized of GLO-1 in serum (FIG. 11B) and in EVs (FIG. 11D) to their respective loading total proteins. Each point represents the value obtained from one patient or control subject. The difference between groups was analyzed with one-way ANOVA followed by the LSD post hoc test. FIG. 11E: Comparison between serum and EVs GLO-1 levels in different study groups. The difference in each group was analyzed with Student-t test. Values are mean±S.E.M with * p<0.05, ** p<0.01, *** p<0.001.

FIGS. 12A-D show the results of ROC curve analysis. The plots represent the performance of RAGE levels in EVs to differentiate LS AD patients to ES and MS AD patients (FIGS. 12A, B) and the performance of GLO-1 levels in EVs to differentiate ES AD patients to MCI and control subjects (FIGS. 12C, D). Area under the curve (AUC) values, 95% confidence intervals (CI 95%), standard error (Std. Error) and p values are indicated on the curve.

FIGS. 13A-J show statistical (Pearson) correlation between RAGE, GFAP and GLO-1 levels and cognitive scores (MMSE and MoCA).

FIGS. 14A-D show statistical (Pearson) correlation between RAGE, GFAP and GLO-1 levels in serum and EVs.

FIGS. 15A-C show the identification of the presence of GLO-1 in neuronal EVs. FIG. 15A: representative Western blot showing the detection of monomeric and dimeric forms of GLO-1 in EVs from AD patients, neuronal EVs and SK-N-SH cells. The Coomassie blue stained total proteins was used as the loading control. FIGS. 15B, C: Comparison between the levels of GLO-1 monomeric (FIG. 15B) and dimeric (FIG. 15C) forms in cells and EVs. The difference in each group was analyzed with Student-t test. Values are mean±S.E.M with * p<0.05.

### DETAILED DESCRIPTION

The use of the terms “a” and “an” and “the” and similar referents in the context of describing the disclosure (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (“e.g.”, “such as”, etc.) provided herein, is intended merely to better illustrate the disclosure and does not pose a limitation on the scope of the disclosure unless otherwise claimed.

No language in the specification should be construed as indicating any non-claimed element as essential to the practice of the disclosure.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Unless otherwise defined, all technical and scientific terms used herein have the same meaning as commonly understood by one of ordinary skill in the art to which this disclosure belongs.

Any and all combinations and subcombinations of the embodiments and features disclosed herein are encompassed by the present disclosure.

In the studies described herein, the present inventors have found that extracellular vesicles (also called exosomes) isolated from plasma (pEVs) are a source of markers for the identification of subjects suffering from mild cognitive impairment (MCI) or early stage Alzheimer's disease (AD), i.e. having a high risk of developing AD. pEVs samples from subjects with MCI and AD were found to contain low levels of Brain-Derived Neurotrophic Factor (BDNF), Neuron Specific Enolase (NSE), S100 calcium-binding protein B (S100B) and Progranulin (PGRN), and a low PGRN/BDNF ratio, relative to control subjects. Furthermore, EVs from subjects suffering from MCI, early and moderate stage of AD had significantly lower levels of receptor for advanced glycation end products (RAGE) relative to last stage AD patients.

Accordingly, in a first aspect, the present disclosure provides method for identifying a subject suffering from mild cognitive impairment (MCI) or early stage Alzheimer's disease (AD), or for identifying a subject having a high risk of developing AD, comprising determining, in a sample comprising extracellular vesicles (EVs) from the subject, at least one of (i) levels of Brain-Derived Neurotrophic Factor (BDNF, UniProtKB accession P23560); (ii) levels of Neuron Specific Enolase (NSE, UniProtKB accession P09104); (iii) levels of S100 calcium-binding protein B (S100B, UniProtKB accession P04271); (iv) levels of Progranulin (PGRN, UniProtKB accession P28799); (v) a ratio of the levels of PGRN to BDNF (PGRN/BDNF ratio); and (vi) levels of glyoxalase 1 (GLO-1, UniProtKB accession Q04760), wherein lower levels of BDNF in the sample relative to a reference BDNF level, lower levels of NSE in the sample relative to a reference NSE level, lower levels of S100B in the sample relative to a reference S100B level, lower levels of PGRN in the sample relative to a reference PGRN level, a lower PGRN/BDNF ratio (or a higher BDNF/PGRN ratio) in the sample relative to a reference PGRN/BDNF ratio and/or lower levels of GLO-1 in the sample relative to a reference GLO-1 level is indicative that the subject suffers from MCI or early stage AD. In an embodiment, the method comprises determining the levels of BDNF. In an embodiment, the method comprises determining the levels of NSE. In an embodiment, the method comprises determining the levels of S100B. In an embodiment, the method comprises determining the levels of PGRN. In an embodiment, the method comprises determining the levels of PGRN and BDNF. In an embodiment, the method comprises determining the PGRN/BDNF ratio. In an embodiment, the method comprises determining the levels of GLO-1. In an embodiment, the levels of BDNF, NSE, S100B, GLO-1 and PGRN are measured concurrently in a multiplex assay. In an embodiment, the levels of BDNF and PGRN are measured concurrently in a multiplex assay. In an embodiment the method comprises determining the levels of BDNF and PGRN in the sample, and calculating the PGRN/BDNF ratio.

Mild cognitive impairment or MCI refers to a mild but noticeable and measurable decline in cognitive abilities, including memory and thinking skills, which is associated with an increased risk of developing AD.

Early stage AD (or mild AD) refers to the first stage of AD that is associated with significant trouble with memory and thinking that impacts daily functioning, which may include memory loss of recent events, difficulty with problem-solving, complex tasks and sound judgments, changes in personality, difficulty organizing and expressing thoughts and/or getting lost or misplacing belongings.

Extracellular vesicles refer to small vesicles (usually 30-200 nm) containing RNA, lipids, metabolites and proteins that are secreted by various types of cells, and found in body fluids including blood, saliva, urine, and breast milk.

In an embodiment, the method is for identifying a subject suffering from MCI, and wherein the levels of BDNF, NSE, S100B and/or PGRN, or the PGRN/BDNF ratio, is determined. In another embodiment, the method is for identifying a subject suffering from early stage AD, and wherein the levels of BDNF, GLO-1, NSE and/or S100B, or the PGRN/BDNF ratio, are determined.

In another aspect, the present disclosure relates to a method for identifying a subject suffering from early stage Alzheimer's disease (AD) comprising determining, in a sample comprising plasma-derived extracellular vesicles (EVs) from the subject, levels of glyoxalase 1 (GLO-1), wherein lower levels of GLO-1 in the sample relative to a reference GLO-1 level, is indicative that the subject suffers from early stage AD.

In another aspect, the present disclosure relates to a method for detecting a change in cognitive function over time in a subject, comprising determining in a sample comprising plasma-derived extracellular vesicles (EVs) from the subject, at least one of: (i) levels of BDNF; (ii) levels of S100B; (iii) levels of PGRN; (iv) levels of RAGE (UniProtKB accession Q15109); (v) levels of GFAP (UniProtKB accession P14136); (vi) levels of GLO-1; and (vii) a ratio of the levels of PGRN to BDNF (PGRN/BDNF ratio); at a first time point and at a second time point, wherein lower levels of PGRN/BDNF ratio, S100B, RAGE, GFAP and/or GLO-1; and/or higher levels of PGRN and/or BDNF at said second time point relative to said first time point is indicative of a decline of cognitive function over time in the subject.

In another aspect, the present disclosure relates to a method of measuring the levels BDNF, NSE, S100B, RAGE, GFAP, GLO-1 and/or PGRN in a sample from a subject (e.g., a subject at risk or suspected of suffering from MCI or early stage AD), comprising (i) obtaining a sample comprising extracellular vesicles (EVs) from the subject; (ii) contacting the sample with one or more reagents that bind to BDNF, NSE, S100B, RAGE, GFAP, GLO-1 and/or PGRN (e.g., a BDNF binding agent, an NSE binding agent, a S100B binding agent, a GLO-1 binding agent, a PGRN binding reagent, a RAGE binding reagent, a GFAP binding reagent or any combination thereof); and (iii) detecting binding between BDNF, NSE, S100B, RAGE, GFAP, GLO-1 and/or PGRN and the one or more reagents.

Methods to measure the amount/level of proteins are well known in the art. Protein levels may be detected directly using a ligand binding specifically to the protein, such as an antibody or an antigen-binding fragment thereof. In embodiments, such a binding molecule or reagent (e.g., antibody or antigen-binding fragment thereof) is labeled/conjugated, e.g., radio-labeled, chromophore-labeled, fluorophore-labeled, or enzyme-labeled to facilitate detection and quantification of the complex (direct detection). Alternatively, protein levels may be detected indirectly, using a binding molecule or reagent, followed by the detection of the [protein/binding molecule or reagent] complex using a second ligand (or second binding molecule) specifically recognizing the binding molecule or reagent (indirect detection). Such a second ligand may be radio-labeled, chromophore-labeled, fluorophore-labeled, or enzyme-labeled to facilitate detection and quantification of the complex. Enzymes used for labeling antibodies for immunoassays are known in the art, and the most widely used are horseradish peroxidase (HRP) and alkaline phosphatase (AP). Examples of binding molecules or reagents include antibodies (monoclonal or polyclonal), natural or synthetic ligands, and the like. In an embodiment, the ligand is an antibody. In embodiment, two antibodies binding to two different epitopes in the proteins (BDNF, NSE, S100B, RAGE, GFAP, GLO-1 and/or PGRN) are used. In an embodiment, the antibody or at least one of the two antibodies is labeled, e.g., radio-labeled, chromophore-labeled, fluorophore-labeled, or enzyme-labeled.

Examples of methods to measure the amount/level of protein in a sample include, but are not limited to: Western blot, immunoblot, enzyme-linked immunosorbent assay (ELISA), “sandwich” immunoassays, radioimmunoassay (RIA), immunoprecipitation, surface plasmon resonance (SPR), chemiluminescence, fluorescent polarization, phosphorescence, immunohistochemical (IHC) analysis, matrix-assisted laser desorption/ionization time-of-flight (MALDI-TOF) mass spectrometry, microcytometry, microarray, antibody array, microscopy (e.g., electron microscopy), flow cytometry, proteomic-based assays, and assays based on a property or activity of the protein including but not limited to ligand binding or interaction with other protein partners, enzymatic activity, fluorescence. For example, if the protein of interest is a kinase known to phosphorylate of given target, the level or activity of the protein of interest may be determined by the measuring the level of phosphorylation of the target in the presence of the test compound. If the protein of interest is a transcription factor known to induce the expression of one or more given target gene(s), the level or activity of the protein of interest may be determined by the measuring the level of expression of the target gene(s). In an embodiment, the amount/level of protein in a sample is measured using an immunoassay, such as an ELISA, e.g., a sandwich ELISA using two specific antibodies binding to different epitopes of BDNF, NSE, S100B, RAGE, GFAP, GLO-1 or PGRN. Commercial reagents and kits for the detection of BDNF, NSE, S100B, RAGE, GFAP, GLO-1 and/or PGRN are available from several providers including ThermoFisher Scientific (Luminex™/ProcartaPlex™ technologies), Abcam, Aviva Systems Biology, Raybiotech, R&D Systems and Novus Biologicals, for example.

“Reference level”, “Control level” or “standard level” are used interchangeably herein and broadly refers to a separate baseline level measured in one or more comparable “control” samples, which may be from subjects not suffering from the disease. The corresponding reference level may be a level corresponding to an average/mean or median level calculated based of the levels measured in several reference or control subjects (e.g., a pre-determined or established standard level). The control level may be a pre-determined “cut-off” value recognized in the art or established based on levels measured in samples from one or a group of control subjects. For example, the “threshold reference level” may be a level corresponding to the minimal level of BDNF, NSE, S100B, RAGE, GFAP, GLO-1 and/or PGRN expression or PGRN/BDNF ratio (cut-off) that permits to distinguish in a statistically significant manner subjects suffering from MCI or early stage AD (or having a high likelihood from suffering from MCI or early stage AD) from those not suffering from MCI or early stage AD (or having a low likelihood from suffering from MCI or early stage AD), which may be determined using samples from MCI or early stage AD patients and from healthy subjects, for example. Alternatively, the “reference level” may be a level corresponding to the level of BDNF, NSE, S100B, RAGE, GFAP, GLO-1 and/or PGRN expression or PGRN/BDNF ratio (cut-off) that permits to best or optimally distinguish in a statistically significant manner subjects suffering from MCI or early stage AD (or having a high likelihood from suffering from MCI or early stage AD) from those not suffering from MCI or early stage AD (or having a low likelihood from suffering from MCI or early stage AD). The corresponding reference/control level may be adjusted or normalized for age, gender, race, or other parameters. The reference level can thus be a single number/value, equally applicable to every patient individually, or the control level can vary, according to specific subpopulations of patients. Thus, for example, older men might have a different control level than younger men, and women might have a different control level than men. The predetermined standard level can be arranged, for example, where a tested population is divided equally (or unequally) into groups, such as a low-risk group, a medium-risk group and a high-risk group or into quadrants or quintiles, the lowest quadrant or quintile being individuals with the lowest risk (i.e., highest level of BDNF, NSE, S100B, RAGE, GFAP, GLO-1 and/or PGRN or PGRN/BDNF ratio) and the highest quadrant or quintile being individuals with the highest risk (i.e., highest level of BDNF, NSE, S100B, RAGE, GFAP, GLO-1 and/or PGRN or PGRN/BDNF ratio). It will also be understood that the control levels according to the disclosure may be, in addition to predetermined levels or standards, levels measured in other samples (e.g. from healthy/normal subjects) tested in parallel with the experimental sample. The reference or control levels may correspond to normalized levels, i.e. reference or control values subjected to normalization based on the level of a housekeeping protein or total protein levels.

In an embodiment, the reference BDNF level is about 80, 78, 75 or 72 pg/mL, as measured by ELISA (e.g., sandwich ELISA). In an embodiment, the reference BDNF level is about 65, 62, 60, 59 or 58 pg/mL, as measured by ELISA (e.g., sandwich ELISA). In an embodiment, the reference NSE level is about 410, 405, 403, 400, 395 or 394 pg/mL, as measured by ELISA (e.g., sandwich ELISA). In an embodiment, the reference S100B level is about 560, 555, 554, 552, 550 or 549 pg/mL, as measured by ELISA (e.g., sandwich ELISA). In an embodiment, the reference PGRN level is about 485, 480 or 475 pg/mL, as measured by ELISA (e.g., sandwich ELISA). In an embodiment, the reference PGRN/BDNF ratio is about 15, 14.8, 14.5, 14.3, 14.1 or 14.

“Lower expression” or “lower level of expression” or “lower levels” as used herein refers to (i) lower levels of BDNF, NSE, S100B, RAGE, GFAP, GLO-1 and/or PGRN or PGRN/BDNF ratio in one or more given cells present in the sample (relative to the control) and/or (ii) lower amount of cells expressing BDNF, NSE, S100B, RAGE, GFAP, GLO-1 and/or PGRN in the sample (relative to the control). In an embodiment, lower refers to a level that is below the reference level (e.g., the predetermined cut-off value). In another embodiment, lower level refers to a level that is at least one standard deviation below the control level (e.g., the predetermined cut-off value) (e.g. that is statistically significant as determined using a suitable statistical analysis). In other embodiments, higher refers to a level of expression that is at least 1.5, 2, 2.5, 3, 4 or 5 standard deviations below the control level (e.g., the predetermined cut-off value. In another embodiment, “lower level” refers to a level that is at least 10, 20, 30, 40 or 50% lower in the test sample relative to the control/reference level. In another embodiment, lower level refers to a level that is at least 1.5, 2-, 5-, or 10-fold lower in the test sample relative to the control/reference level (e.g., the predetermined cut-off value).

In an embodiment, the above-mentioned method comprises a step of normalizing the protein levels, i.e. normalization of the measured levels of the above-noted proteins against a standard, for example the total protein content in the sample or the level of a stably expressed control protein (or housekeeping protein) to facilitate the comparison between different samples. “Normalizing” or “normalization” as used herein refers to the correction of raw protein level values/data between different samples for sample to sample variations, to take into account differences in “extrinsic” parameters such as cellular input, protein quality, purification, etc., i.e. differences not due to actual “intrinsic” variations in protein levels in the samples. Such normalization is performed by correcting the raw protein level values/data for a test protein (BDNF, NSE, S100B, RAGE, GFAP, GLO-1 and/or PGRN) based on the protein level values/data measured for one or more “housekeeping” or “control” proteins, i.e. whose levels are known to be constant (i.e. to show relatively low variability) under different experimental conditions, or the total protein content in the sample. Thus, in an embodiment, the above-mentioned method further comprises measuring the level of a housekeeping protein or the total protein content in the biological sample (e.g., pEVs), and normalizing the protein level values/data for the test protein (BDNF, NSE, S100B, RAGE, GFAP, GLO-1 and/or PGRN) based on the levels of the housekeeping protein or the total protein content.

In another embodiment, the method described herein further comprises obtaining or collecting a biological sample comprising EVs from a subject. In various embodiments, the sample can be from any source that contains EVs, for example a blood or blood-derived sample such as plasma. In an embodiment, the EVs are plasma EVs (pEVs). Thus, in an embodiment, the method described herein further comprise a step of isolating pEVs (or enrichment of pEVs) from the plasma sample obtained from the subject. Thus, the sample may be subjected to purification/enrichment techniques to obtain a sample enriched in EVs (e.g., pEVs). Accordingly, in an embodiment, the method may be performed on an isolated EV (e.g., pEV) sample. Methods and kits for purification of EVs (exosomes) are well known in the art (Tang et al., Int J Mol Med. 2017, 40(3): 834-844), and include ultracentrifugation (UC)-based purification methods, as well as commercially available systems such as the Total Exosome Isolation Kit/Reagents from Invitrogen/ThermoFisher Scientific, the qEV EV/exosome isolation system from Izon Science Ltd., and the ExoQuick™ Exosome Isolation kit series from System Biosciences. The methods described herein may further include step(s) for enriching for CNS-derived EVs, for example by immunoaffinity using a reagent (e.g., antibody) capable of binding to a molecule expressed by CNS-derived EVs, such as L1-cell adhesion molecule (L1CAM). In an embodiment, the sample comprises at least 10% of EVs (e.g., pEVs). In other embodiments, the sample comprises at least 20%, 30%, 40%, 50%, 60%, 70%, 80%, 90% or 95% of EVs (e.g., pEVs).

The biological sample may be collected using any methods for collection of biological fluid, tissue or cell sample, such as venous puncture for collection of blood samples.

In certain embodiments, the methods described herein may be at least partly, or wholly, performed in vitro. In a further embodiment, the method is wholly performed in vitro.

In an embodiment, the above-mentioned method further comprises selecting and/or administering a course of therapy or prophylaxis to a subject suffering from MCI or early stage AD identified using the method described herein. For example, if it is determined that the subject has MCI or early stage AD, the subject may be subjected to a therapy for AD to delay the onset and/or progression of AD.

In an embodiment, the above-mentioned method further comprises monitoring over time the cognitive status of subject suffering from MCI or early stage AD identified using the method for detecting a change (e.g., decline) in cognitive function over time in a subject described herein.

In another aspect, the present disclosure relates to a method for preventing or delaying a decline of cognitive function in a subject, the method comprising administering a therapy for improving cognitive function in a subject having a decline in cognitive function identified using the method

In an embodiment, the above-mentioned method further comprises discontinuing cognitively impairing medications in the subject suffering from MCI or early stage AD identified using the method described herein. Cognitively impairing medications include certain anticholinergics, as well as certain cardiovascular agents such as antihypertensives, diuretics and antiarrhythmics.

Thus, in another aspect, the present disclosure relates to a method for preventing, or delaying the onset or progression of AD, of a subject suffering from MCI or early stage AD, comprising identifying said subject suffering from MCI or early stage AD using the method described herein, and administering a course of therapy or prophylaxis for AD to the subject to prevent, or delay the onset or progression of AD, in the subject.

Examples of therapy for AD include cholinesterase inhibitors such as Razadyne® (galantamine), Exelon® (rivastigmine), and Aricept® (donepezil), as well as N-methyl D-aspartate (NMDA) antagonists such as Namenda® (memantine). The therapy may also include physical and/or cognitive exercise that has been shown to significantly improve cognitive measures (Smith, J. C. et al. 2013. Journal of Alzheimer's Disease, 37 (1), 197-215; Petersen et al., Neurology, Jan. 16, 2018; 90(3)).

Assessment of cognitive function may be made using commonly used tests such as the 7-Minute Screen, Mini-Cog, the Memory Impairment Screen, the Short Test of Mental Status, the Abbreviated Mental Test, the 6-Item Screener, the Hopkins Verbal Learning Test, the 6-Item Cognitive Impairment Test, the Clock Drawing Test, DemTect, Mini-Mental State Exam (MMSE) and the Montreal Cognitive Assessment (MoCA) (see, e.g., Andrew N. Wilner, Neurology Reviews. 2008 September; 16(9):5). In an embodiment, assessment of cognitive function is performed using MMSE. In an embodiment, assessment of cognitive function is performed using MoCA. In an embodiment, assessment of cognitive function is performed using both MMSE and MoCA.

In another aspect, the present disclosure provides an assay mixture for the assessment of MCI or early stage AD (e.g., for identifying a subject suffering from MCI or early stage AD), the assay mixture comprising: (i) a sample comprising EVs from a subject (e.g., a subject suspected or at risk of suffering from MCI or early stage AD; and (ii) one or more reagents for determining/measuring the level of BDNF, NSE, S100B, GLO-1 and/or PGRN in the sample.

In another aspect, the present disclosure provides an assay mixture for detecting a change in cognitive function over time in a subject, the assay mixture comprising: (i) a sample comprising EVs from a subject (e.g., a subject suspected or at risk of suffering from MCI or early stage AD; and (ii) one or more reagents for determining/measuring the level of BDNF, NSE, S100B, RAGE, GFAP, GLO-1 and/or PGRN in the sample.

In another aspect, the present disclosure provides a system for the assessment of MCI or early stage AD (e.g., for identifying a subject suffering from MCI or early stage AD), the system comprising (i) a sample comprising EVs from a subject (e.g., a subject suspected or at risk of suffering from MCI or early stage AD; and (ii) one or more reagents for determining/measuring the level of BDNF, NSE, S100B, GLO-1 and/or PGRN in the sample. In an embodiment, the sample is an isolated EV (e.g., pEV) sample.

In another aspect, the present disclosure provides a system for detecting a change in cognitive function over time in a subject, the system comprising (i) a sample comprising EVs from a subject (e.g., a subject suspected or at risk of suffering from MCI or early stage AD; and (ii) one or more reagents for determining/measuring the level of BDNF, NSE, S100B, RAGE, GFAP, GLO-1 and/or PGRN in the sample. In an embodiment, the sample is an isolated EV (e.g., pEV) sample.

In another aspect, the present disclosure provides a system for the assessment of MCI or early stage AD (e.g., for identifying a subject suffering from MCI or early stage AD), the system comprising a sample analyzer configured to produce one or more signals corresponding to the levels of one or more of BDNF, NSE, S100B and/or PGRN in a sample comprising EVs of the subject; and a computer sub-system programmed to calculate whether the one or more signal(s) is/are lower than corresponding reference value(s). In various embodiments, the system further comprises the sample, for example an isolated EV (e.g., pEV) sample.

In another aspect, the present disclosure provides a system for detecting a change in cognitive function over time in a subject, the system comprising a sample analyzer configured to produce one or more signals corresponding to the levels of one or more of BDNF, NSE, S100B, RAGE, GFAP, GLO-1 and/or PGRN in a sample comprising EVs of the subject; and a computer sub-system programmed to calculate whether the one or more signal(s) is/are lower and/or higher than corresponding reference value(s). In various embodiments, the system further comprises the sample, for example an isolated EV (e.g., pEV) sample.

In another aspect, the present disclosure provides a kit for identifying a subject suffering from MCI or early stage AD comprising: (a) reagents for determining the levels of BDNF, NSE, S100B and/or PGRN; and (b) instructions setting forth the method for identifying a subject suffering from MCI or early stage AD described herein.

In another aspect, the present disclosure provides a kit for identifying a subject suffering from early stage AD comprising (a) reagents for determining the levels of GLO-1; and (b) instructions setting forth the method for identifying a subject suffering from early stage AD as described herein.

In another aspect, the present disclosure provides a kit for detecting a change in cognitive function over time in a subject comprising (a) reagents for determining the levels of BDNF, S100B, PGRN, RAGE, GFAP and/or GLO-1; and (b) instructions setting forth the method for detecting a change in cognitive function over time in a subject as described herein.

In an embodiment, the one or more reagents comprise, for example, antibodies specific for BDNF, NSE, S100B, PGRN, RAGE, GFAP and/or GLO-1, secondary antibodies, reagents for detecting antigen-antibody complexes (e.g., enzymatic substrates), etc.

Furthermore, in an embodiment, the kit may be divided into separate packages or compartments containing the respective reagent components explained above.

In addition, such a kit may optionally comprise one or more of the following: (1) instructions for using the reagents for (i) the identification a subject suffering from MCI or early stage AD or (ii) detecting a change in cognitive function over time in a subject according to the methods described herein; (2) one or more containers; and/or (3) appropriate controls/standards. Such a kit can include reagents for collecting a biological sample from a patient and reagents for processing the biological sample, for example for enriching the sample in EVs. The kits featured herein can also include an instruction sheet describing how to perform the assays for measuring BDNF, NSE, S100B, PGRN, RAGE, GFAP and/or GLO-1 levels. The instruction sheet can also include instructions for how to determine a reference cohort (control patient population), including how to determine expression levels in the reference cohort and how to assemble the expression data to establish a reference for comparison to a test patient. The instruction sheet can also include instructions for assaying protein levels in a test patient and for comparing the levels with the level in the reference cohort to determine whether the subject suffers from MCI or early stage AD, and undertake an appropriate treatment regimen for the test patient if needed.

Informational material included in the kits can be descriptive, instructional, marketing or other material that relates to the methods described herein and/or the use of the reagents for the methods described herein. For example, the informational material of the kit can contain contact information, e.g., a physical address, email address, website, or telephone number, where a user of the kit can obtain substantive information about performing the method described herein and interpreting the results, particularly as they apply to determining whether the subject suffers from MCI or early stage AD.

The kits featured herein can also contain software necessary to infer a patient's likelihood of having MCI or early stage AD from the BDNF, NSE, S100B, GLO-1 and/or PGRN levels measured in the sample, or to infer a patient's likelihood of having a change (e.g., decline) in cognitive function from the BDNF, S100B, GLO-1, RAGE, GFAP and/or PGRN levels measured in the sample.

In another aspect, there is provided the use of the kit or assay mixture described herein for the identification of a subject suffering from MCI or early stage AD, or for the identification of a subject having a change (e.g., decline) in cognitive function over time.

### EXAMPLES

The present disclosure is illustrated in further details by the following non-limiting examples.

### Example 1: Materials and Methods

Selection of Participants (Examples 1 to 5)

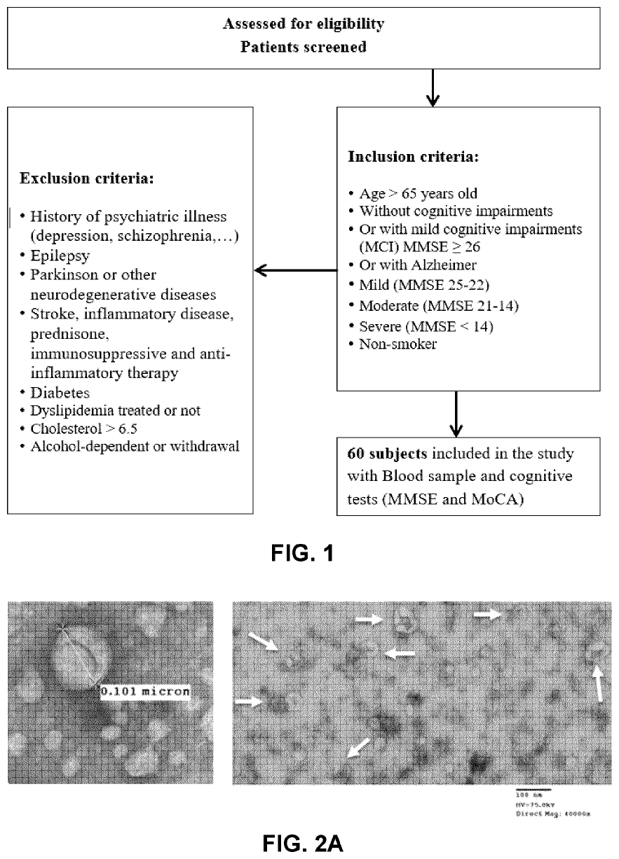

Plasma samples were obtained from 60 participants recruited from the Memory Clinic of Sherbrooke including control subjects, MCI and AD patients at different stages (mild, moderate and severe). The table 1 lists characteristics of patients and controls. MCI subjects were clinically diagnosed with criteria of Petersen (20) and the different stages of AD were detected by applying clinical criteria of the National Institute of Neurological and Communicative Disorders and Stroke and the Alzheimer's Disease and Related Disorders Association (NINCDS-ADRDA) (6). Control subjects were defined according to the SENIEUR protocol (from SENIor EURopean), a standard selection protocol for immunogerontological studies (21). The inclusion and exclusion criteria of the study are listed in FIG. 1.

Descriptive statistics on age, gender, MMSE and MoCA scores of the subject are shown in Table 1. The number of participants was equal in each group with a higher female predominance. Scores of MMSE and MoCA tests were significantly lower in AD groups compared to control subjects. Unlike MMSE scores, MoCA scores were reduced earlier in MCI group because of their greater sensitivity for MCI detection (23). Age was significantly lower in controls than in other groups but these differences were considered in the present analyses.

TABLE 1
Characteristics of patients with MCI, AD and control participants

| | | Gender | | MMSE | MoCA |
|---|---|---|---|---|---|
| | | (male/ | Age | scores | scores |
| Diagnosis | n | female) | (years) | (/30 points) | (/30 points) |
| Controls | 12 | 3/9 | 68.8 ± 1.5 | 29.4 ± 0.3 | 28.1 ± 0.5 |
| MCI | 12 | 1/11 | 75.3 ± 1.2 ^{a} | 27.9 ± 0.3 | 22.4 ± 1.1 ^{b} |
| Mild AD | 12 | 1/11 | 75.6 ± 1.3 ^{b} | 24.0 ± 0.5 ^{c} | 19.7 ± 1.5 ^{c} |
| Moderate AD | 12 | 4/8 | 79.1 ± 1.1 ^{c} | 19.9 ± 1.4 ^{c} | 14.0 ± 0.9 ^{c} |
| Severe AD | 12 | 2/10 | 83.0 ± 1.6 ^{c} | n.d. | n.d. |

Values are expressed as means ± standard error of the mean (SEM). Statistical analysis was performed using the one way ANOVA followed by Tukey post hoc tests (alpha = 0.05),
^{a} p < 0.05,
^{b} p < 0.01,
^{c} p < 0.001 compared to healthy controls.
Abbreviations: MCI, mild cognitive impairment; MMSE, mini-mental state examination; MoCA, Montreal cognitive assessment; n.d., not determined.

Study Population and Collection of Samples (Examples 6 to 10)

Venous blood samples were collected from control subjects (n=10), MCI (n=10) and different stage of AD patients (early stage (n=10), moderate stage (n=10) and late stage (n=10)). Blood was centrifugated to collect serum fraction that were stored at −80° C. until analysis. Patients recruitment was performed by the Memory Clinic of Sherbrooke and a written informed consent was provided prior to blood and data collection. The Mini Mental State Examination (MMSE) and the Montreal Cognitive Assessment (MoCA) was administered to examine global cognitive function of all participants (Folstein, M. F. et al. J Psychiatr Res 1975, 12, 189-198; Nasreddine, Z. S. et al. J Am Geriatr Soc 2005, 53, 695-699). Control subjects were defined according to the SENIEUR protocol (Pawelec, G. et al. Mech Ageing Dev 2001, 122, 132-134). Cognitive test scores and Pertersen criteria was performed for MCI selection (Petersen et al. Arch Neurol 1999, 56, 303-308). The criteria of the National Institute of Neurological and Communicative Disorders and Stroke and the Alzheimer's Disease and Related Disorders Association (NINCDS_ADRDA) and the fourth edition of the Diagnostic and Statistical Manual of Mental Disorders (DSM-IV) were used for AD patient selection (McKhann, G. Neurology 1984, 34, 939-944). The clinical characteristics of the study population is presented in Table 2.

Significant differences were observed on the mean age scores between control subjects, MCI and the three stages of AD patients. MMSE scores were significantly lower in AD groups but not in MCI patients compared to control subjects. However, the mean and standard error of MoCA scores were significantly lower in MCI patients and AD groups compared to control subjects. LS (AD) patients were not able to answer or complete the questions of MMSE and MoCA tests.

TABLE 2
Clinical characteristics of study population

| | | Gender | | MMSE | MoCA |
|---|---|---|---|---|---|
| Param- | | (male/ | Age | scores | scores |
| eters | n | female) | (years) | (/30 points) | (/30 points) |
| Con- | 10 | 2/8 | 69.6 ± 1.6 | 29.4 ± 0.3 | 27.8 ± 0.6 |
| trols | | | | | |
| MCI | 10 | 1/9 | 76.8 ± 0.6*** | 27.4 ± 0.5 | 22.6 ± 1.3*** |
| ES AD | 10 | 0/10 | 76.3 ± 1.4*** | 24.7 ± 0.7*** | 19.3 ± 1.5*** |
| MS AD | 10 | 3/7 | 79.4 ± 1.1*** | 19.4 ± 1.3*** | 13.2 ± 1.2*** |
| LS AD | 10 | 2/8 | 82.4 ± 1.5*** | n.d. | n.d. |

Statistical analysis was performed using the one-way ANOVA followed by LSD test with
* P < 0.05,
** P < 0.01,
***P < 0.001 versus control subjects.
Values are mean ± standard error of the mean (SEM).
Abbreviations: AD: Alzheimer disease; ES: early stage of Alzheimer disease; MS: Moderate-stage of Alzheimer disease; LS: late-stage of Alzheimer disease; MMSE. Mini-Mental State Examination; MoCA. Montreal Cognitive Assessment; ND: not detected.

Cognitive Assessments and Plasma Collection

From all patients, blood was obtained after overnight fasting in heparin-containing vacuum tubes and immediately separated by low speed centrifugation at 260×g for 15 minutes (22° C.). The plasma was aliquoted and stored at −80° C. until used to avoid freeze/thaw cycles. Global cognitive function was assessed by the MoCA and the MMSE scores from all patients except severe AD(18, 19). Prior to isolation of pEVs, plasma samples were diluted at 1:2 in filtered Phosphate Saline Buffer (PBS) and were centrifuged at 2000×g for 20 minutes followed by a second centrifugation at 10,000×g for 20 minutes to remove cells and cell debris.

Isolation of Total Extracellular Vesicles from Plasma and Neuronal 416 SK-N-SH Culture Media

The clarified plasma samples and SK-N-SH culture media were precipitated using the Total Exosome Isolation reagent (Invitrogen™ by Life Technologies Inc., Carlsbad, Calif., USA) during 30 min at 4° C. After centrifugation at 10,000×g for 5 minutes, pEVs pellets were re-suspended in filtered PBS and purified by three series of filtrations (100 KDa)/precipitations. Final solutions of pEVs were re-suspended and conserved in filtered PBS at −80° C. for further analysis.

Characterization of Total Extracellular Vesicles from Plasma: TEM, NTA and Western Blot Analysis

The method for isolation of total pEVs was validated by various approaches.

The shape and the size of pEVs isolated were visualized using transmission electron microscopy (TEM). Final solutions of pEVs were suspended in 2% paraformaldehyde and 10 pL of the mixture were adsorbed for 5 minutes to a Formvar-carbon coated grid. Grids were negatively stained using 2% uranyl acetate solution for 1 minute. After excess uranyl formate was removed with filter paper and grids were examined using HITACHI® 7100 transmission electron microscope (75 kV) at 15000×-40000× magnification.

Then, the size distribution and the concentration of isolated pEVs were measured by the Nanosight® NS300 system and the Nanosight® NTA 3.2 Analytical Software (Malvern Instruments Company, Nanosight®, and Malvern, United Kingdom). Recordings were performed for 60 seconds and the measurement was conducted three times for each sample. Prior to injection in the chamber of the NanoSight® using a sterile syringe, pEVs suspensions were diluted in filtered PBS at 1:2000.

Finally, the absence of a negative control (Calnexin) and the presence of some EVs markers (TSG101, GAPDH and CD63) and cerebral markers (L1CAM, GFAP) were confirmed by Western blot analysis. The total proteins of pEVs were extracted using Radio Immuno Precipitation Assay (RIPA) buffer (50 mM Tris buffer, pH 8, 150 mM sodium chloride, 0.1% sodium dodecyl sulfate, 1% Igepal, 1% sodium deoxycholate, 5 mM EDTA, 1% protease and phosphatase inhibitor cocktail) and their concentrations were quantified using bicinchoninic acid (BCA) assay (Pierce™ BCA Protein Assay Kit, ThermoFisher Scientific, Inc). The same pEVs protein amount (15 μg) was separated using 10% SDS-PAGE gel and transferred to PVDF membranes. The membranes were blocked in Tris-buffered saline containing 0.1% Tween™ 20 (TBS-T) and 5% nonfat dry milk before incubation (overnight at 4° C.) with the following primary antibodies: TSG101 (MBS7605273, MyBiosource, Inc, San Diego, Calif., USA); CD63 (Sc-5275, Santa Cruz, Biotechnologies, Santa Cruz, Calif., USA); Calnexin (Sc-, Santa Cruz, Biotechnologies, Santa Cruz, Calif., USA); L1CAM (Sc-53386, Santa Cruz, Biotechnologies, Santa Cruz, Calif., USA); GFAP (G9269, EMD Millipore Corp., Burlington, Mass., USA). Then, membranes were washed with TBS-T and HRP-conjugated secondary antibodies were incubated for 1 h at room temperature (7076S, Anti-mouse IgG HRP-linked Antibody or 7074S, Anti-rabbit HRP-linked Antibody from Cell Signaling Technology, Inc., Danvers, Mass., USA). The membrane blots were detected by chemiluminescence using ECL substrate (Bio-Rad Laboratories, Inc., Hercules, Calif., USA) and the FluorChem HD2 system. The membranes were stained with Coomassie blue to detect the profile of EVs total proteins.

Quantification of Brain Derived Proteins: Multianalyte Immunoassay

A Luminex™ assay was performed to measure the concentrations of BDNF, NSE, Progranulin and S100B in 50 μL of extracted pEVs according to supplier's directions (R&D Systems, Inc., Minneapolis, Minn., USA). Assay sensitivities (minimum detectable concentrations in pg/ml) were 0.32, 140.00, 195.00 and 4.34 for BDNF, NSE, Progranulin and S100B, respectively. The assay was run with the Luminex 100/200 and data were analyzed using Xponent™ 4.2 software. Marker values were normalized with the total protein quantification in pEVs.

Evaluation of GFAP and GLO-1 Levels

The levels of GFAP and GLO-1 were evaluated by western blot. Briefly, 20 pg of serum or EVs protein were heated at 95° C. in the presence of loading buffer. The mixture was separated using 10% SDS-PAGE. After proteins separation, gel was transferred to PVDF membranes and the membranes were blocked for 1 h at room temperature with TBS containing 5% BSA. Then, each membrane was incubated with correspondent primary antibody GFAP (1/5000) (EMD Millipore, MA, USA) GLO-1 (1/2000) (MyBiosourse Inc, San Diego, Calif. USA) in TBS with 5% BSA and incubated overnight at 4° C. After 3 washes for 5 min, HRP-conjugated anti-rabbit antibody (1/2000) (Cell Signaling Technologie) were incubated in TBS with 5% BSA. Finally, the membrane was washed 3 times for 5 min and were visualized by chemiluminescence detection using ECL substrate (Biorad) and their level was analyzed with the luminescent imaging system FluorChem™. Total proteins stained with Coomassie blue were used as a loading control.

To evaluate the presence and the amount of GLO-1 in neuronal SK-N-SH cells and EVs, total proteins from SK-N-SH cells and EVs were extracted with RIPA buffer containing a cocktail of protease and phosphatase inhibitors and were measured using BCA assay. The same proteins amount (20 pg) were used to in western blot to determine GLO-1 as described above.

RAGE Assay

The levels of serum EVs RAGE was measured using a Luminex assay (R&D Systems, Inc., USA) according to the manufacturer's instructions. Other proteins were measured in the same plate, but data will be used for a future study. Diluted sample from all participants were added to pre-coated beads with specific human RAGE antibodies. After the addition of biotinylated detection antibodies and phycoerythrin (PE)-conjugated streptavidin, beads were read using the Luminex 100/200 and data were analyzed using Xponent 4.2 software. Concentrations of RAGE in EVs were normalized with the total protein amount.

Statistical Analysis

SPSS or GraphPad Prism program were used to perform data analysis. Data are presented as mean±SEM. P value less than 0.05 was considered statistically significant. After using the Shapiro-Wilk test to verify normal distribution, the statistical significance of differences between groups were determined by the one-way analysis of variance (ANOVA) followed by the Tukey or LSD post-hoc test and Student t-test. For not normally distributed data including Progranulin levels, we performed the nonparametric Kruskal-Wallis test followed by Dunn's test to determine significant differences between groups. Correlation analyses were performed using the Pearson correlation coefficient. Receiver operating characteristics (ROC) curves were constructed and the area under the ROC curve (AUC) was calculated to determine the ability of a marker to discriminate between the diseased and control populations (22). The ROC analysis provided also the diagnostic sensitivity and specificity of each markers (Prism 7.04).

### Example 2: Characterization of Total Extracellular Vesicles Isolated from Plasma

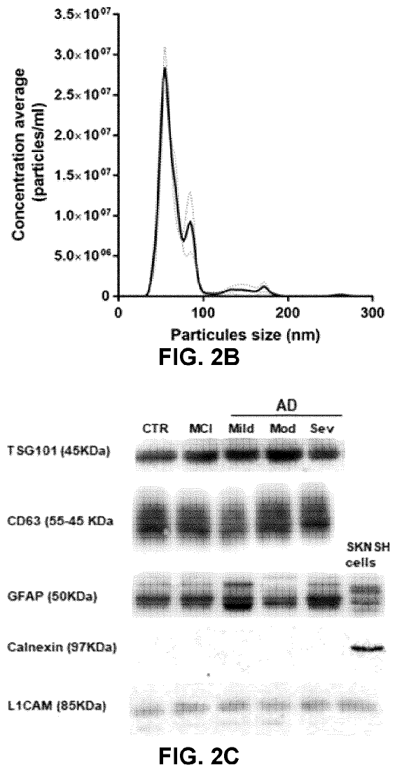

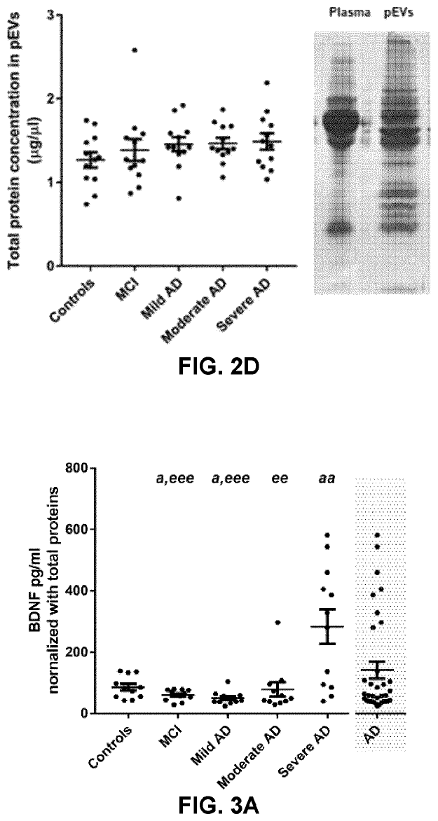

Different approaches were used to confirm the presence of pEVs. After isolation, the pEVs were morphologically characterized with TEM and the cup-shaped morphology, characteristic of EVs, could be observed (FIG. 2A).

In addition, the size distribution of the pEVs population was analyzed using a platform for nanoparticle characterization (Nanosight® NS300). Thus, it was confirmed that most of the pEVs had a size smaller than 200 nm and that the pEVs size was mainly ranged between 40 and 100 nm (FIG. 2B).

Then, lysates from isolated pEVS were separated by SDS/PAGE and analyzed by immunoblot to verify the presence of common EVs markers like TSG101 and CD63 (FIG. 2C). Western blot analyses revealed the presence of brain-derived EVs in pEVs with the existence of neuronal and glial markers (L1CAM and GFAP, respectively), suggesting the presence of some CNS-derived EVs in the pEVs samples. Finally, Calnexin, a negative EVs marker, was absent in EVs but was present in SK-N-SH cells. Interestingly, the levels of EVs and cerebral markers in pEVs did not vary significantly among the different group of subjects.

The total protein content of pEVs was determined. No significant difference was measured between different group of subjects, suggesting that disease progression does not affect the total protein components in pEVs, and thus that this parameter could be used to normalize the levels of specific proteins measured in the pEVs samples (FIG. 2D). FIG. 2D also shows that the total protein profile was different between plasma and isolated pEVs.

### Example 3: Brain-Derived Proteins Levels in Total Circulating Extracellular Vesicles

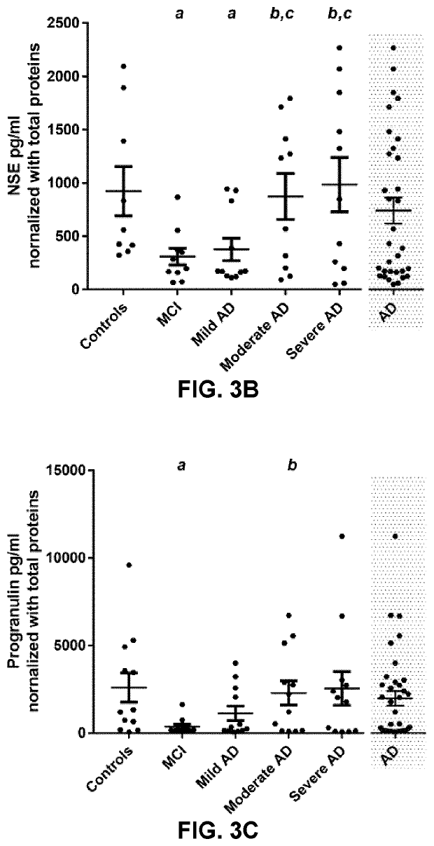

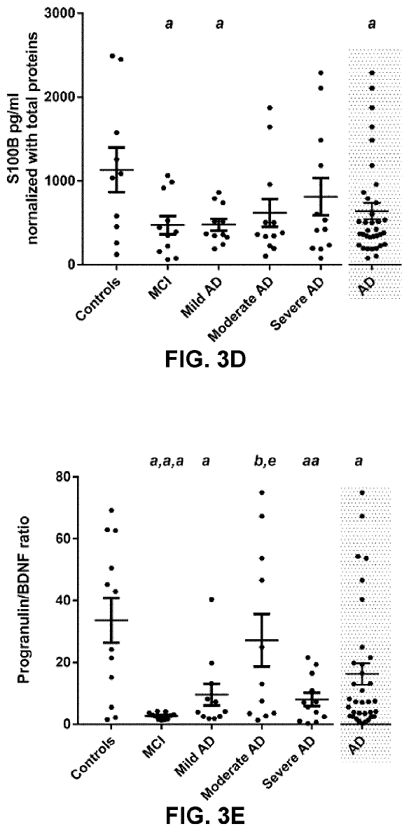

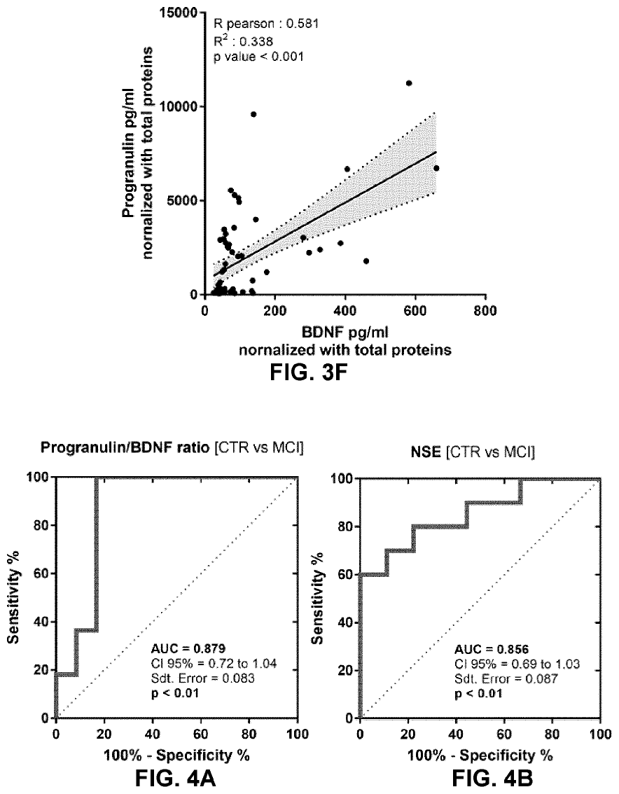

BDNF, NSE, Progranulin and S100B levels were significantly lower in pEVs of MCI subjects relative to the control group (FIG. 3A-D). The decrease in BDNF, NSE and S100B levels was also observed in pEVs from mild AD patients. For BDNF levels in pEVs, an increase in the terminal stage of AD was detected. By contrast, there was no difference in the levels of the other markers in pEVs from patients at the moderate and severe stages of AD relative to control participants. This suggests that these four proteins could be useful for the detection of MCI and mild AD patients.

### Example 4: Accuracy of Brain-Derived Proteins Levels in Total Circulating Extracellular Vesicles for Identifying MCI Subjects

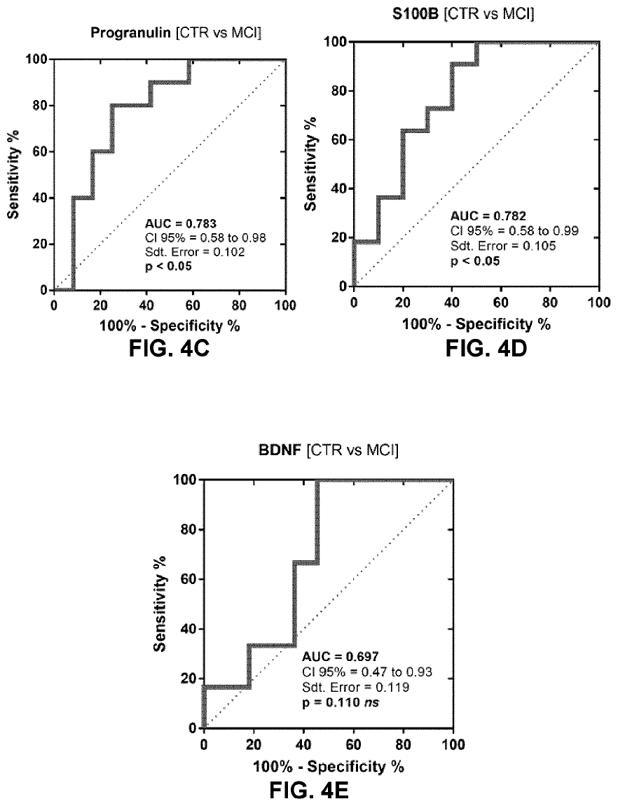

The accuracy of each brain-derived protein for identifying MCI subjects was investigated using receiver-operating characteristic curves (ROC). The area under the curve (AUC) provided the discriminatory ability of brain-derived proteins with 95% of confidence intervals. To distinguish the control group from MCI patients, AUC for the Progranulin/BDNF ratio and NSE levels in pEVs were superior to 0.80, indicating high classification accuracy (FIGS. 4A, 4B). Progranulin and S100B levels offered a good discrimination between the MCI group and control participants with AUC of 0.783 and 0.782, respectively (FIGS. 4C, 4D). In contrast, levels of BDNF in pEVs provided a poor differentiation power between MCI patients and control group with an AUC of 0.697 (FIG. 4E).

ROC analyses also provided the optimal cutoff and the sensitivity and specificity of the brain-derived protein levels, as summarized in Table 3. According to the criteria proposed by the National Institute on Aging (NIA), the ideal AD biomarker should have a sensitivity and specificity greater than 80% (26). The results indicated that the Progranulin/BDNF ratio in pEVs could be a strong indicator of MCI with a sensitivity of 90.9% and a specificity of 83.3%. Levels of NSE and Progranulin in pEVs provided a sensibility of 80% and a specificity superior to 75%.

TABLE 3
Cutoff values to discriminate control
participants (CTR) from MCI subjects

| Biomarker | Cutoff | Sensitivity | Specificity |
|---|---|---|---|
| (CTR vs MCI) | (pg/mL) | (%) | (%) |
| Progranulin/BDNF ratio | <4.1 | 90.9 | 83.3 |
| NSE | <394.0 | 80.0 | 77.8 |
| Progranulin | <475.0 | 80.0 | 75.0 |
| S100B | <554.0 | 72.7 | 70.0 |
| BDNF | <71.3 | 66.7 | 63.6 |

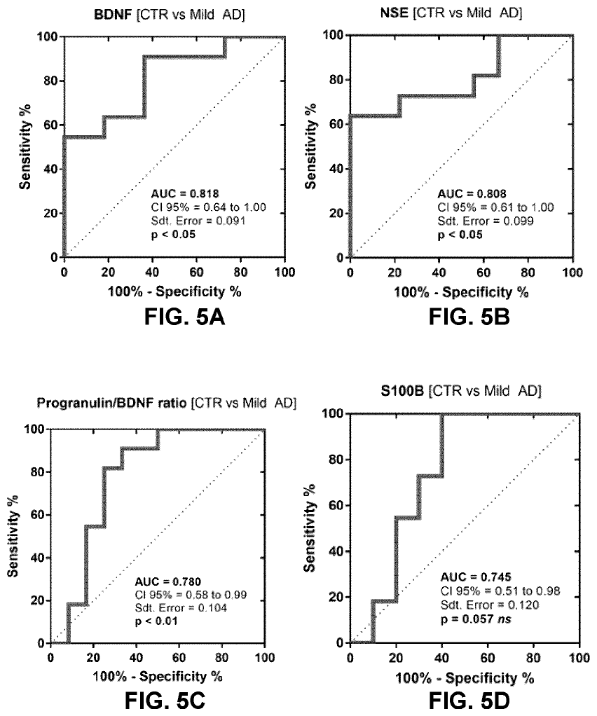

It was also found that levels of BDNF and NSE in pEVs could be considered as robust markers to distinguish control participants from mild AD patients with AUC of 0.818 and 0.808, respectively (FIG. 5A, 5B). The ratio of Progranulin/BDNF and levels of S100B were less efficient to discriminate patients with mild stage AD from control subjects with AUC of 0.780 and 0.745, respectively (FIG. 5C, 5D).

Table 4 reports the sensitivity and specificity of each marker for the discrimination of control participants and mild AD patients. The ratio of Progranulin/BDNF in pEVs corresponded to the best marker with sensitivity and specificity of 81.8% and 75%, respectively. The levels of NSE and S100B had lowest efficiency with AUC inferior to 75%.

TABLE 4
Cutoff values to discriminate control
participants from mild AD patients

| Biomarker | Cutoff | Sensitivity | Specificity |
|---|---|---|---|
| (CTR vs MCI) | (pg/mL) | (%) | (%) |
| Progranulin/BDNF ratio | <14.2 | 81.8 | 75 |
| NSE | <403.0 | 72.7 | 77.8 |
| Progranulin | ns | — | — |
| S100B | <549.0 | 72.7 | 70.0 |
| BDNF | <58.1 | 72.7 | 63.6 |

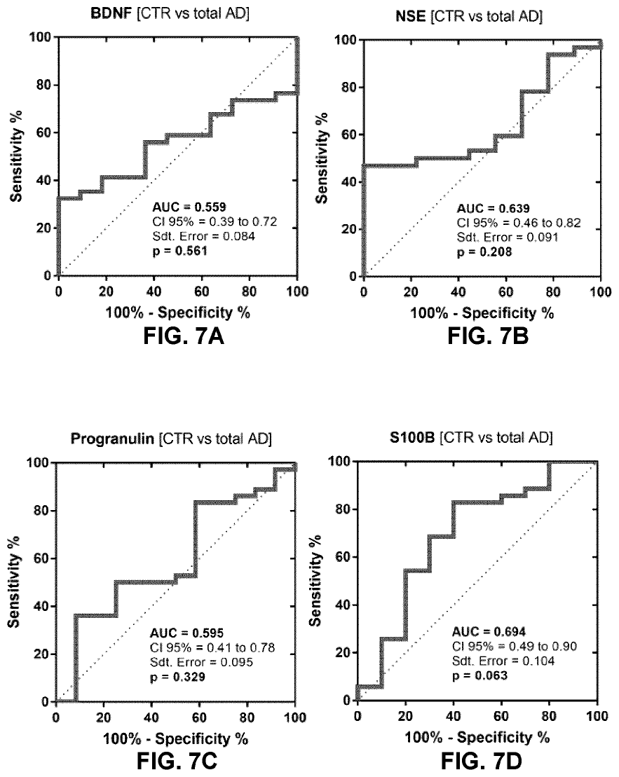

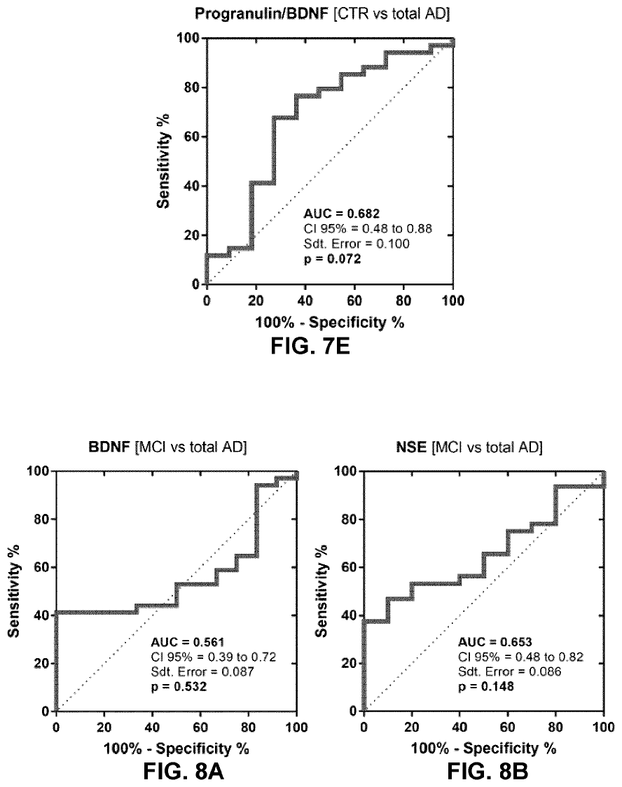

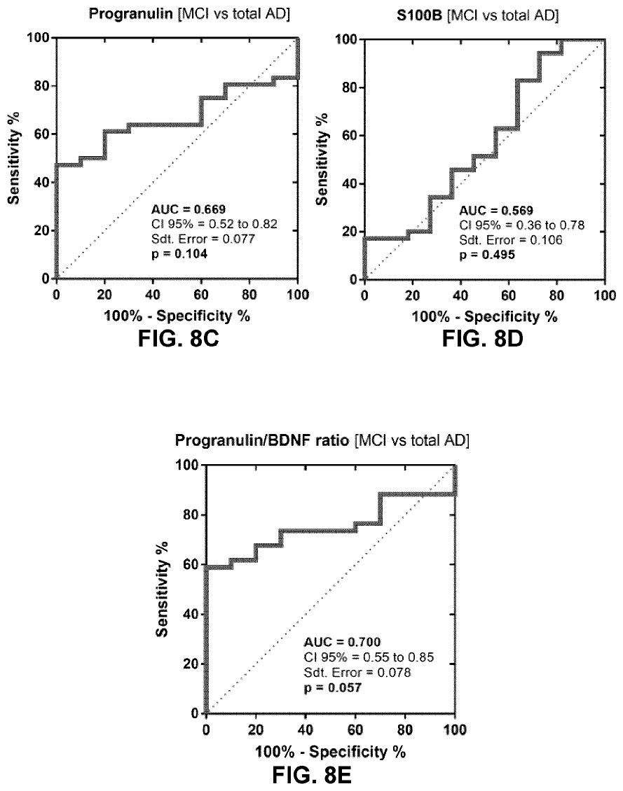

The ratio of Progranulin/BDNF and levels of BDNF, NSE, Progranulin and S100B in pEVs provided a poor differentiation power (AUC of 0.682, 0.559, 0.639, 0.595 and 0.694, respectively), for the discrimination between control subjects and AD patients when all stages were pooled (FIGS. 7A-7E), or for the discrimination between MCI subjects from AD patients (all stages combined) (AUC of 0.700, 0.561, 0.653, 0.669 and 0.569, respectively) (FIGS. 8A-8E).

### Example 5: Relationship Between Brain-Derived Protein Levels to Cognitive Performance

According to the criteria proposed by the Alzheimer's disease neuroimaging initiative (ADNI), the relationship between the ideal biomarker and a disease parameter meaningful to the patient, as cognitive function, should be clearly established (27). Therefore, the relationship between scores of two cognitive tests (MMSE and MoCa) and levels of brain-derived proteins in pEVs was examined.

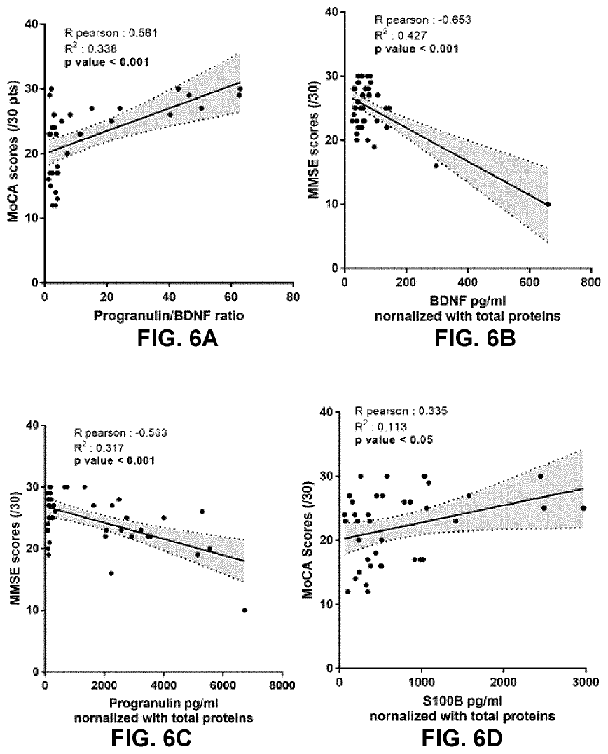

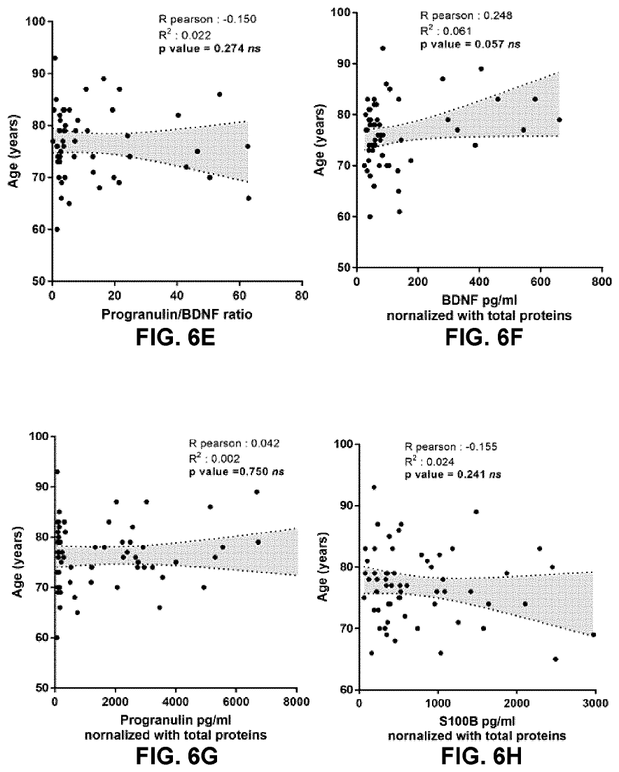

A strong positive correlation was observed between cognitive performance, assessed by MoCA, and the ratio of Progranulin/BDNF in pEVs (FIG. 6A). A negative correlation between the cognitive function (evaluated by MMSE test) and levels of BDNF and Progranulin, individually, was also measured (FIGS. 6B, 6C). Finally, there was also a positive correlation between S100B concentrations in pEVs and MoCA scores (FIG. 6D). To note, the levels of these proteins in pEVs were not age-related (p=0.274, p=0.057, p=0.750, p=0.241 for Progranulin/BDNF ratio, BDNF, Progranulin and S100B, respectively).

### Example 6: RAGE Levels in Peripheral EVs

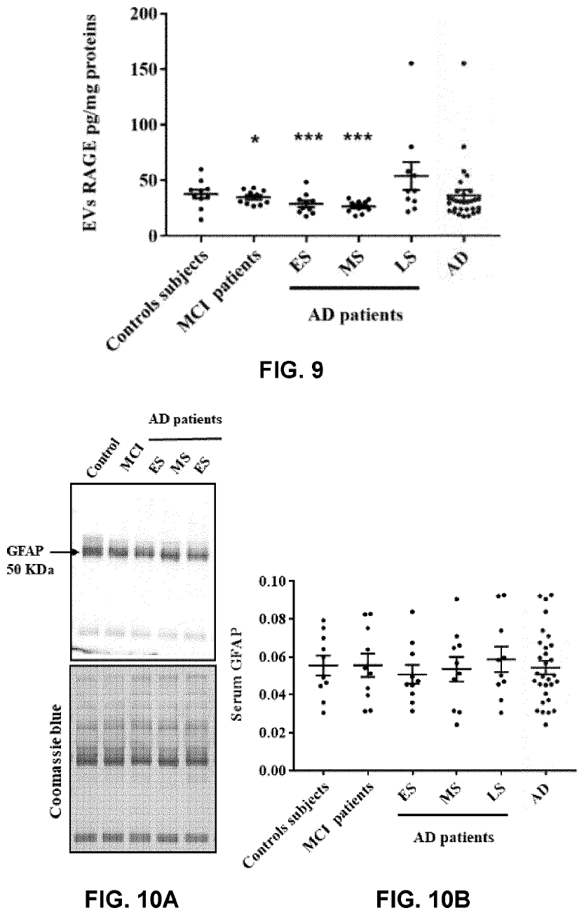

Circulating EVs were isolated from control subjects, MCI and AD patients. Luminex assay using specific antibody was performed to detect and quantify RAGE levels. The results showed that peripheral EVs contained RAGE. Moreover, RAGE levels were significantly lower in MCI group, early and moderate stage of AD patients relative to the last stage of AD patients (FIG. 9).

### Example 7: GFAP and GLO-1 Levels in Serum and EVs

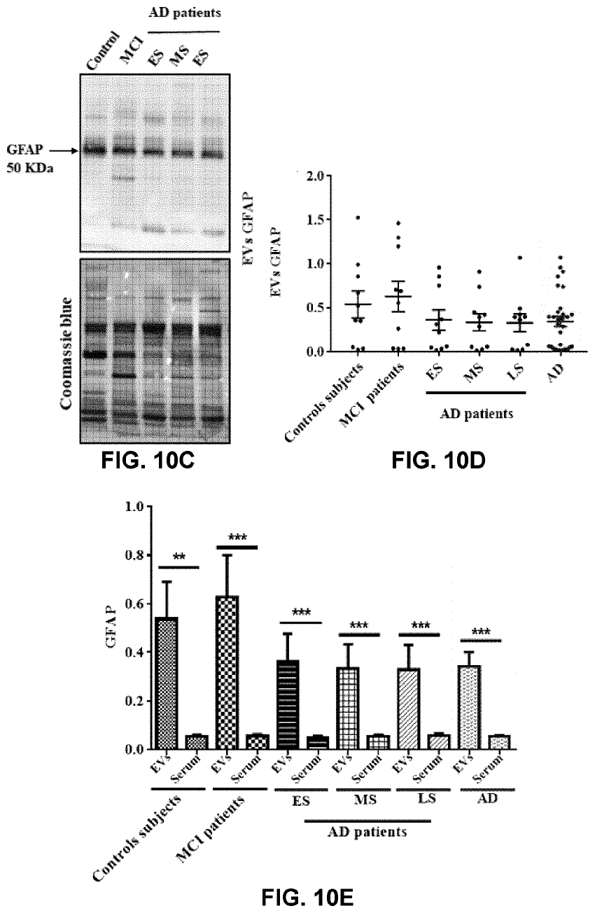

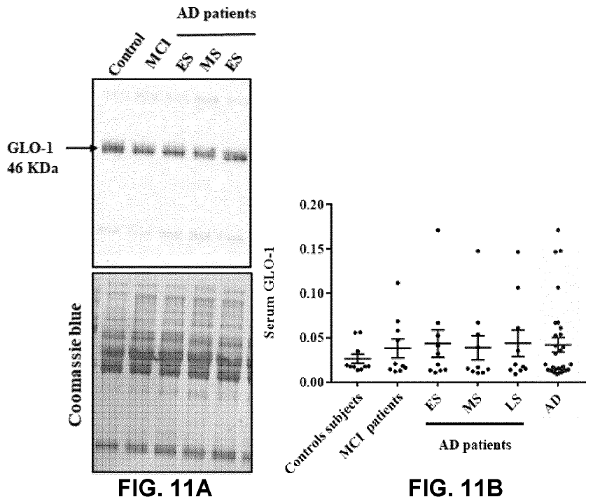

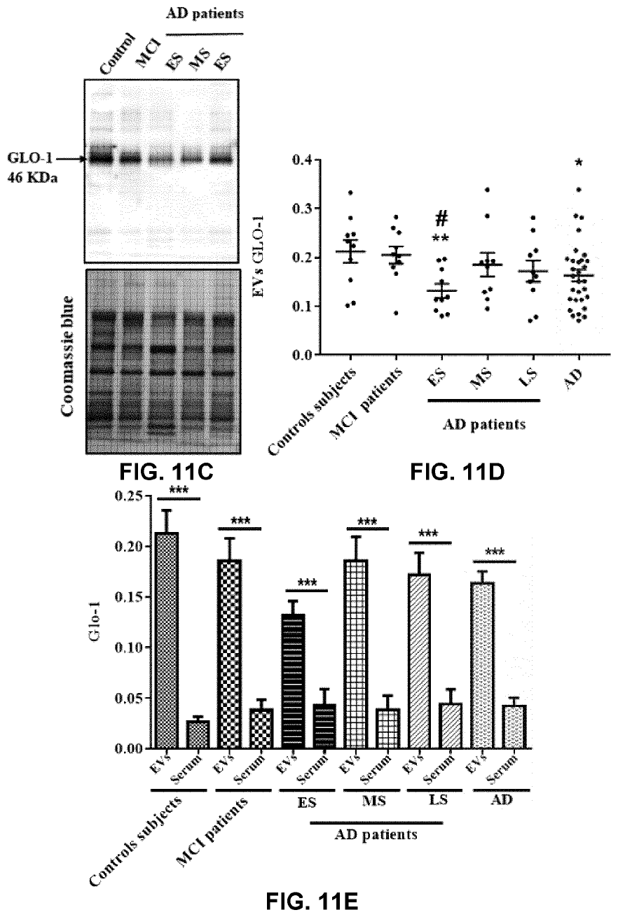

The presence of GFAP and GLO-1 in serum and peripheral EVs and their levels were assessed in control subjects, MCI and AD patients by Western blot. The results show that GFAP and GLO-1 are both present in serum and EVs. Molecular weight of GFAP detected in serum and EVs is 50 KDa. There is no significant difference in the serum or EVs GFAP levels between control subjects, MCI and all AD's groups (FIGS. 10A-D). Interestingly, the comparison between GFAP levels in serum and EVs show that the vesicles amount of this protein is higher than in serum (FIG. 10E). Dimeric form of GLO-1 (46 KDa) was detected in serum and EVs of all groups (FIGS. 11A-C). GLO-1 levels in serum were not statistically different among five groups (FIG. 11B). However, EVs GLO-1 levels were significantly decreased in AD group and specifically in early AD group relative to control subjects and MCI patients (FIG. 11D). Comparison between GLO-1 levels in serum and EVs show that the amount of this protein is higher in vesicles than in serum (FIG. 11E).

### Example 8: RAGE and GLO-1 Levels in EVs Differentiate Stage of AD

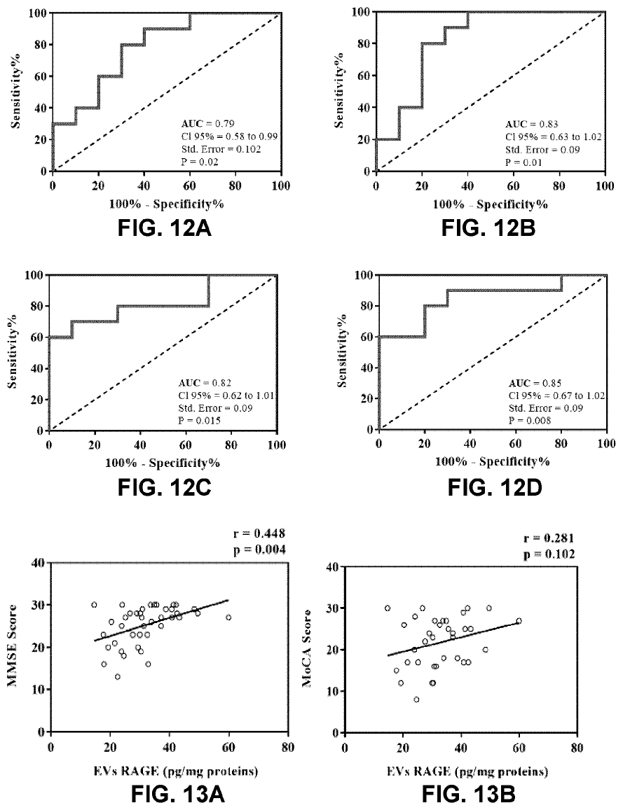

The ability of the levels of RAGE and GLO-1 in EVs to distinguish different AD stage from MCI patients and control subjects was assessed using ROC analysis. The levels EVs RAGE provide a fair classification of the LS AD patients from ES and MS AD patients with an area under the curve (AUC) of 0.79 (95% CI: 0.58-0.99, p=0.02) and 0.83 (95% CI: 0.63-1.02, p=0.01), respectively (FIGS. 12A, B). To distinguish ES AD patients from control subjects and MCI patients, ROC curves for EVs GLO-1 levels show high classification accuracy with an AUC of 0.82 (95% CI: 0.62-1.01, p=0.015) and 0.85 (95% CI: 0.67-1.02, p=0.008), respectively (FIGS. 12C, D).

### Example 9: Correlation Between RAGE, GAFAP and GLO-1 Levels and Cognitive Scores

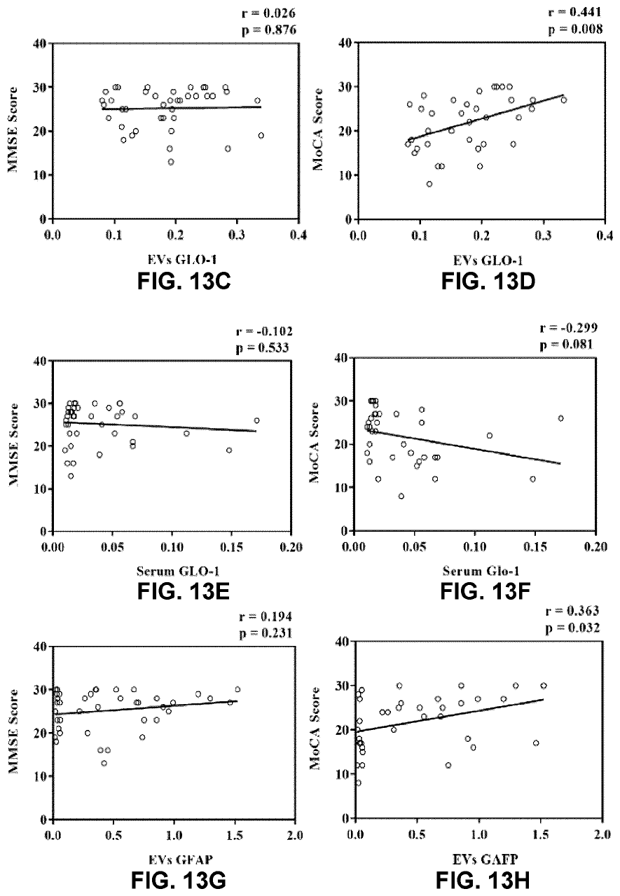

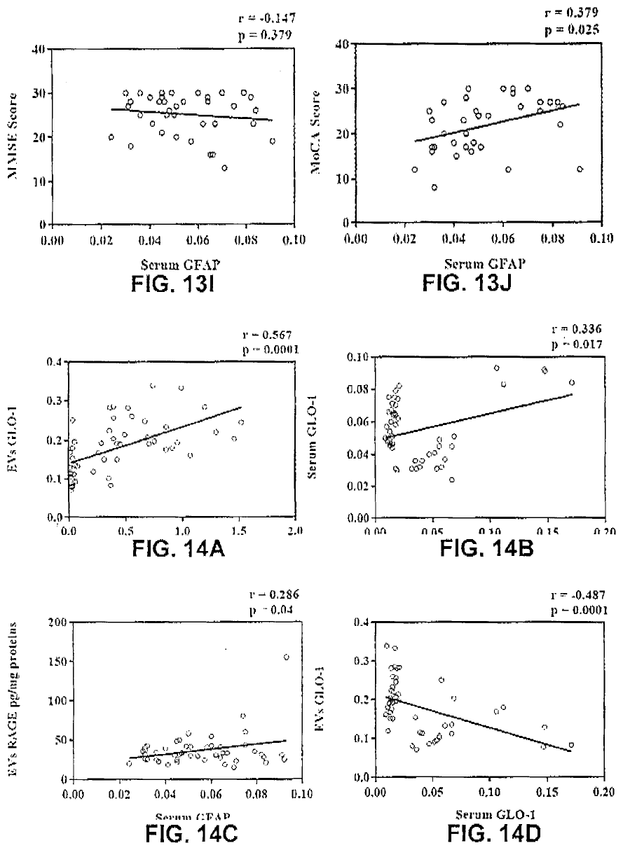

Pearson correlation was used to evaluated eventual correlation between RAGE, GFAP and GLO-1 levels and cognitive scores (MMSE and MoCA). It was found that RAGE levels in EVs correlate with MMSE but not with MoCA score (FIGS. 13A, B). GLO-1 levels in EVs were shown to correlate with MoCA but not with MMSE scores (FIGS. 13C, D). However, there was no correlation between serum GLO-1 levels and both MMSE and MoCA scores (FIGS. 13E, F). GFAP levels in serum and EVs were also shown to be correlated with MoCA scores (FIGS. 13G-J). It was confirmed that there was no significative correlation between the age of the subjects and any of the markers (RAGE, GFAP, GLO-1).

A positive correlation between GLO-1 and GFAP levels in EVs and serum was observed (FIGS. 14A, B) and between EVs RAGE levels and serum GFAP levels (FIG. 14C). A negative correlation between GLO-1 levels in EVs and serum was shown (FIG. 14D). No other correlation between markers levels was found.

### Example 10: Detection of GLO-1 in Neuronal EVs

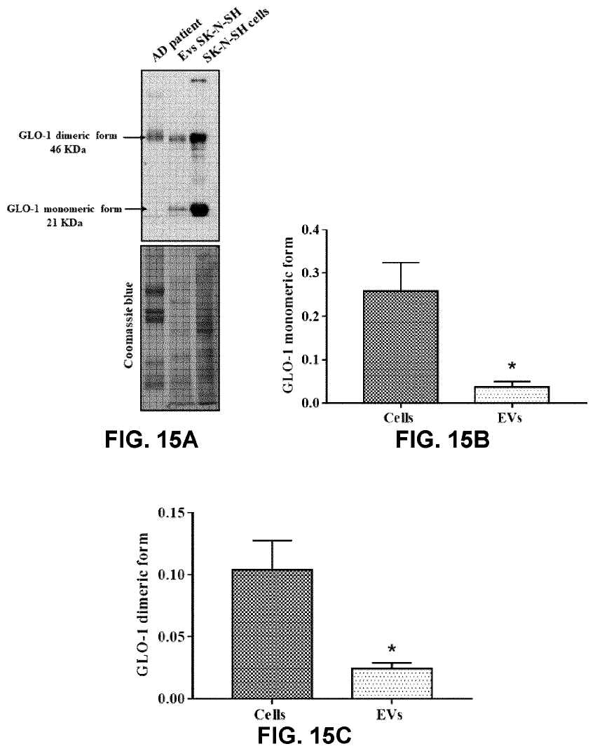

To identify the presence of GLO-1 in neuronal EVs, SK-N-SH cells were used to EVs isolation. The presence of two forms of GLO-1 was detected in neuronal EVs, a monomeric form (21 KDa) and a dimeric form (46 KDa). These two forms are also present in neuronal SK-N-SH neuronal cells, but not in peripheral EVs from patients in which only the dimeric form was detected (FIG. 15A). Comparison between cells and neuronal EVs GLO-1 monomeric and dimeric forms revealed that neurons can release low amounts of GLO-1 in EVs relative to the total cellular GLO-1 amount (FIG. 15B, C).

Although the present disclosure has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Holsinger R M, Schnarr J, Henry P, Castelo V T, Fahnestock M. Quantitation of BDNF mRNA in human parietal cortex by competitive reverse transcription-polymerase chain reaction: decreased levels in Alzheimer's disease. Brain research Molecular brain research. 2000; 76(2):347-54.
2. Chitramuthu B P, Bennett H P J, Bateman A. Progranulin: a new avenue towards the understanding and treatment of neurodegenerative disease. Brain: a journal of neurology. 2017; 140(12):3081-104.
3. Van Eldik L J, Griffin W S. S100 beta expression in Alzheimer's disease: relation to neuropathology in brain regions. Biochimica et biophysica acta. 1994; 1223(3):398-403.
4. Baig S, Palmer L E, Owen M J, Williams J, Kehoe P G, Love S. Clusterin mRNA and protein in Alzheimer's disease. Journal of Alzheimer's disease: JAD. 2012; 28(2):337-44.
5. Sperling R A, Aisen P S, Beckett L A, Bennett D A, Craft S, Fagan A M, et al. Toward defining the preclinical stages of Alzheimer's disease: recommendations from the National Institute on Aging-Alzheimer's Association workgroups on diagnostic guidelines for Alzheimer's disease. Alzheimer's & dementia: the journal of the Alzheimer's Association. 2011; 7(3):280-92.
6. Dubois B, Feldman H H, Jacova C, Dekosky S T, Barberger-Gateau P, Cummings J, et al. Research criteria for the diagnosis of Alzheimer's disease: revising the NINCDS-ADRDA criteria. The Lancet Neurology. 2007; 6(8):734-46.
7. Coleman B M, Hill A F. Extracellular vesicles—Their role in the packaging and spread of misfolded proteins associated with neurodegenerative diseases. Seminars in cell & developmental biology. 2015; 40:89-96.
8. Rajendran L, Honsho M, Zahn T R, Keller P, Geiger K D, Verkade P, et al. Alzheimer's disease beta-amyloid peptides are released in association with exosomes. Proceedings of the National Academy of Sciences of the United States of America. 2006; 103(30):11172-7.
9. Dinkins M B, Dasgupta S, Wang G, Zhu G, Bieberich E. Exosome reduction in vivo is associated with lower amyloid plaque load in the SXFAD mouse model of Alzheimer's disease. Neurobiology of aging. 2014; 35(8):1792-800.
10. Polanco J C, Scicluna B J, Hill A F, Gotz J. Extracellular Vesicles Isolated from the Brains of rTg4510 Mice Seed Tau Protein Aggregation in a Threshold-dependent Manner. The Journal of biological chemistry. 2016; 291(24):12445-66.
11. Mustapic M, Eitan E, Werner J K, Jr., Berkowitz S T, Lazaropoulos M P, Tran J, et al. Plasma Extracellular Vesicles Enriched for Neuronal Origin: A Potential Window into Brain Pathologic Processes. Frontiers in neuroscience. 2017; 11:278.
12. Shi M, Liu C, Cook T J, Bullock K M, Zhao Y, Ginghina C, et al. Plasma exosomal alpha-synuclein is likely CNS-derived and increased in Parkinson's disease. Acta neuropathologica. 2014; 128(5):639-50.
13. Fiandaca M S, Kapogiannis D, Mapstone M, Boxer A, Eitan E, Schwartz J B, et al. Identification of preclinical Alzheimer's disease by a profile of pathogenic proteins in neurally derived blood exosomes: A case-control study. Alzheimer's & dementia: the journal of the Alzheimer's Association. 2015; 11(6):600-7 e1.
14. Yew D T, Li W P, Webb S E, Lai H W, Zhang L. Neurotransmitters, peptides, and neural cell adhesion molecules in the cortices of normal elderly humans and Alzheimer patients: a comparison. Experimental gerontology. 1999; 34(1):117-33.
15. Gillian A M, Brion J P, Breen K C. Expression of the neural cell adhesion molecule (NCAM) in Alzheimer's disease. Neurodegeneration: a journal for neurodegenerative disorders, neuroprotection, and neuroregeneration. 1994; 3(4):283-91.
16. Smogeli E, Davidson B, Cvancarova M, Holth A, Katz B, Risberg B, et al. L1 CAM as a prognostic marker in stage I endometrial cancer: a validation study. BMC cancer. 2016; 16:596.
17. Markovic-Lipkovski J, Zivotic M, Muller C A, Tampe B, Cirovic S, Vjestica J, et al. Variable Expression of Neural Cell Adhesion Molecule Isoforms in Renal Tissue: Possible Role in Incipient Renal Fibrosis. PloS one. 2015; 10(9):e0137028.
18. Folstein M F, Folstein S E, McHugh P R. “Mini-mental state”. A practical method for grading the cognitive state of patients for the clinician. Journal of psychiatric research. 1975; 12(3):189-98.
19. Nasreddine Z S, Phillips N A, Bedirian V, Charbonneau S, Whitehead V, Collin I, et al. The Montreal Cognitive Assessment, MoCA: a brief screening tool for mild cognitive impairment. Journal of the American Geriatrics Society. 2005; 53(4):695-9.
20. Petersen R C, Smith G E, Waring S C, Ivnik R J, Tangalos E G, Kokmen E. Mild cognitive impairment: clinical characterization and outcome. Archives of neurology. 1999; 56(3):303-8.
21. Ligthart G J, Corberand J X, Fournier C, Galanaud P, Hijmans W, Kennes B, et al. Admission criteria for immunogerontological studies in man: the SENIEUR protocol. Mechanisms of ageing and development. 1984; 28(1):47-55.
22. Soreide K. Receiver-operating characteristic curve analysis in diagnostic, prognostic and predictive biomarker research. Journal of clinical pathology. 2009; 62(1):1-5.
23. Trzepacz P T, Hochstetler H, Wang S, Walker B, Saykin A J. Relationship between the Montreal Cognitive Assessment and Mini-mental State Examination for assessment of mild cognitive impairment in older adults. BMC geriatrics. 2015; 15:107.
24. Poon W W, Blurton-Jones M, Tu C H, Feinberg L M, Chabrier M A, Harris J W, et al. beta-Amyloid impairs axonal BDNF retrograde trafficking. Neurobiology of aging. 2011; 32(5):821-33.
25. Petoukhov E, Fernando S, Mills F, Shivji F, Hunter D, Krieger C, et al. Activity-dependent secretion of progranulin from synapses. Journal of cell science. 2013; 126(Pt 23):5412-21.
26. Consensus report of the Working Group on: “Molecular and Biochemical Markers of Alzheimer's Disease”. The Ronald and Nancy Reagan Research Institute of the Alzheimer's Association and the National Institute on Aging Working Group. Neurobiology of aging.

1998; 19(2):109-16.
27. Mueller S G, Weiner M W, Thal L J, Petersen R C, Jack C R, Jagust W, et al. Ways toward an early diagnosis in Alzheimer's disease: the Alzheimer's Disease Neuroimaging Initiative (ADNI). Alzheimer's & dementia: the journal of the Alzheimer's Association. 2005; 1(1):55-66.
28. Ray S, Britschgi M, Herbert C, Takeda-Uchimura Y, Boxer A, Blennow K, et al. Classification and prediction of clinical Alzheimer's diagnosis based on plasma signaling proteins. Nature medicine. 2007; 13(11):1359-62.
29. O'Bryant S E, Xiao G, Barber R, Reisch J, Doody R, Fairchild T, et al. A serum protein-based algorithm for the detection of Alzheimer disease. Archives of neurology. 2010; 67(9):1077-81.
30. Zhang R, Barker L, Pinchev D, Marshall J, Rasamoelisolo M, Smith C, et al. Mining biomarkers in human sera using proteomic tools. Proteomics. 2004; 4(1):244-56.
31. Van Damme P, Van Hoecke A, Lambrechts D, Vanacker P, Bogaert E, van Swieten J, et al. Progranulin functions as a neurotrophic factor to regulate neurite outgrowth and enhance neuronal survival. The Journal of cell biology. 2008; 181(1):37-41.
32. Matsuda N, Lu H, Fukata Y, Noritake J, Gao H, Mukherjee S, et al. Differential activity-dependent secretion of brain-derived neurotrophic factor from axon and dendrite. The Journal of neuroscience: the official journal of the Society for Neuroscience. 2009; 29(45):14185-98.
33. Tapia L, Milnerwood A, Guo A, Mills F, Yoshida E, Vasuta C, et al. Progranulin deficiency decreases gross neural connectivity but enhances transmission at individual synapses. The Journal of neuroscience: the official journal of the Society for Neuroscience. 2011; 31(31):11126-32.
34. Sheng J G, Mrak R E, Rovnaghi C R, Kozlowska E, Van Eldik L J, Griffin W S. Human brain S100 beta and S100 beta mRNA expression increases with age: pathogenic implications for Alzheimer's disease. Neurobiology of aging. 1996; 17(3):359-63.
35. Marshak D R, Pesce S A, Stanley L C, Griffin W S. Increased S100 beta neurotrophic activity in Alzheimer's disease temporal lobe. Neurobiology of aging. 1992; 13(1):1-7.
36. Mrak R E, Sheng J G, Griffin W S. Correlation of astrocytic S100 beta expression with dystrophic neurites in amyloid plaques of Alzheimer's disease. Journal of neuropathology and experimental neurology. 1996; 55(3):273-9.
37. Chaves M L, Camozzato A L, Ferreira E D, Piazenski I, Kochhann R, Dall'Igna O, et al. Serum levels of S100B and NSE proteins in Alzheimer's disease patients. Journal of neuroinflammation. 2010; 7:6.
38. Cutler N R, Kay A D, Marangos P J, Burg C. Cerebrospinal fluid neuron-specific enolase is reduced in Alzheimer's disease. Archives of neurology. 1986; 43(2):153-4.

1. A method for identifying a subject suffering from mild cognitive impairment (MCI) or early stage Alzheimer's disease (AD) comprising determining, in a sample comprising extracellular vesicles (EVs), preferably plasma-derived extracellular vesicles (pEVs) from the subject, at least one of:
(i) levels of Brain-Derived Neurotrophic Factor (BDNF);
(ii) levels of Neuron Specific Enolase (NSE);
(iii) levels of S100 calcium-binding protein B (S100B);
(iv) levels of Progranulin (PGRN);
(v) a ratio of the levels of PGRN to BDNF (PGRN/BDNF ratio); and
(vi) levels of glyoxalase 1 (GLO-1)
wherein lower levels of BDNF in the sample relative to a reference BDNF level, lower levels of NSE in the sample relative to a reference NSE level, lower levels of S100B in the sample relative to a reference S100B level, lower levels of PGRN in the sample relative to a reference PGRN level, a lower PGRN/BDNF ratio in the sample relative to a reference PGRN/BDNF ratio, and/or lower levels of GLO-1 in the sample relative to a reference GLO-1 level, is indicative that the subject suffers from MCI or early stage AD.

2. The method of claim 1, comprising determining the levels of BDNF.

3. The method of claim 1 or 2, comprising determining the levels of NSE.

4. The method of any one of claims 1 to 3, comprising determining the levels of S100B.

5. The method of any one of claims 1 to 4, comprising determining the levels of PGRN.

6. The method of any one of claims 1 to 5, comprising determining the PGRN/BDNF ratio.

7. The method of any one of claims 1 to 6, comprising determining the levels of GLO-1.

8. The method of any one of claims 1 to 7, wherein the reference BDNF level is about 72 pg/mL.

9. The method of claim 8, wherein the reference BDNF level is about 58.1 pg/mL.

10. The method of any one of claims 1 to 9, wherein the reference NSE level is about 403 pg/mL.

11. The method of claim 10, wherein the reference NSE level is about 394 pg/mL.

12. The method of any one of claims 1 to 11, wherein the reference S100B level is about 554 pg/mL.

13. The method of claim 12, wherein the reference S100B level is about 549 pg/mL.

14. The method of any one of claims 1 to 13, wherein the reference PGRN level is about 475 pg/mL.

15. The method of any one of claims 1 to 14, wherein the reference PGRN/BDNF ratio is about 14,2.

16. The method of claim 15, wherein the reference PGRN/BDNF ratio is about 4,1.

17. The method of any one of claims 1 to 16, further comprising isolating the EVs from a biological sample prior to said determining.

18. The method of any one of claims 1 to 17, wherein the method is for identifying a subject suffering from MCI.

19. The method of any one of claims 1 to 17, wherein the method is for identifying a subject suffering from early stage AD.

1-47. (canceled)

48. A method for preventing or delaying the onset and/or progression of Alzheimer's Disease (AD), the method comprising:
(a) identifying a subject suffering from MCI or early stage AD using a method comprising:
determining, in a sample comprising plasma-derived extracellular vesicles (pEVs) from the subject, at least one of:
(i) levels of Brain-Derived Neurotrophic Factor (BDNF);
(ii) levels of Neuron Specific Enolase (NSE);
(iii) levels of S100 calcium-binding protein B (S100B);
(iv) levels of Progranulin (PGRN);
(v) a ratio of the levels of PGRN to BDNF (PGRN/BDNF ratio); and
(vi) levels of glyoxalase 1 (GLO-1)
wherein the subject is identified as suffering from MCI or early stage AD if:
lower levels of BDNF relative to a reference BDNF level,
lower levels of NSE relative to a reference NSE level,
lower levels of S100B relative to a reference S100B level,
lower levels of PGRN relative to a reference PGRN level,
a lower PGRN/BDNF ratio relative to a reference PGRN/BDNF ratio, and/or
lower levels of GLO-1 relative to a reference GLO-1 level,
are measured in the sample; and
(b) administering a therapy for improving cognitive function or treating AD to the subject.

49. The method of claim 48, comprising determining the levels of BDNF.

50. The method of claim 48, comprising determining the levels of NSE.

51. The method of claim 48, comprising determining the levels of S100B.

52. The method of claim 48, comprising determining the levels of PGRN.

53. The method of claim 48, comprising determining the PGRN/BDNF ratio.

54. The method of claim 48, comprising determining the levels of GLO-1.

55. The method of claim 48, wherein the reference BDNF level is about 72 pg/mL;
the reference NSE level is about 403 pg/mL; the reference S100B level is about 554 pg/mL; the reference PGRN level is about 475 pg/mL; and/or the reference PGRN/BDNF ratio is about 14,2.

56. The method of claim 55, wherein the reference BDNF level is about 58.1 pg/mL.

57. The method of claim 55, wherein the reference NSE level is about 394 pg/mL.

58. The method of claim 55, wherein the reference S100B level is about 549 pg/mL.

59. The method of claim 55, wherein the reference PGRN/BDNF ratio is about 4.1.

60. The method of claim 48, further comprising isolating the pEVs from a biological sample prior to said determining.

61. The method of claim 48, wherein the subject suffers from MCI.

62. The method of claim 48, wherein the subject suffers from early stage AD.

63. A system for identifying a subject suffering from mild cognitive impairment (MCI) or early stage Alzheimer's disease (AD) comprising:
(a) a sample comprising plasma-derived extracellular vesicles (pEVs) from a subject suspected or at risk of suffering from MCI or early stage AD; and
(b) reagents for determining the levels of BDNF, NSE, S100B, GLO-1 and/or PGRN; and
(b) instructions setting forth a method for identifying a subject suffering from MCI or early stage AD comprising determining, in the sample comprising pEVs from the subject, at least one of:
(i) levels of Brain-Derived Neurotrophic Factor (BDNF);
(ii) levels of Neuron Specific Enolase (NSE);
(iii) levels of S100 calcium-binding protein B (S100B);
(iv) levels of Progranulin (PGRN);
(v) a ratio of the levels of PGRN to BDNF (PGRN/BDNF ratio); and
(vi) levels of glyoxalase 1 (GLO-1)
wherein the subject is identified as suffering from MCI or early stage AD if:
lower levels of BDNF relative to a reference BDNF level,
lower levels of NSE relative to a reference NSE level,
lower levels of S100B relative to a reference S100B level,
lower levels of PGRN relative to a reference PGRN level,
a lower PGRN/BDNF ratio relative to a reference PGRN/BDNF ratio, and/or
lower levels of GLO-1 relative to a reference GLO-1 level,
are measured in the sample.

64. The system of claim 63, wherein the reagents for determining the levels of BDNF, NSE, S100B and/or PGRN comprise an anti-BDNF antibody, an anti-NSE antibody, an anti-S100B antibody, an anti-GLO-1 antibody and/or an anti-PGRN antibody.

65. The system of claim 63, further comprising reagents for isolating extracellular vesicles from a plasma sample.

66. The system of claim 63, further comprising a sample analyzer configured to produce one or more signals corresponding to the levels of one or more of BDNF, NSE, S100B and/or PGRN in the sample comprising EVs of the subject; and a computer sub-system programmed to calculate whether the one or more signal(s) is/are lower than corresponding reference value(s).
